(19) United States
(12) Patent Application Publication
(10) Pub. No.: US 2016/0340362 A1
(43) Pub. Date: Nov. 24, 2016

(54) Process For The Preparation Of Oxymorphone Freebase

(71) Applicant: Noramco, Inc., Athens, GA (US)

(72) Inventors: Timothy Samuel Bailey, Blackstone Heights (AU); Paul Jonathan Nichols, Watkinsville, GA (US); Joshua S. Sasine, Bishop, GA (US); Ulrich Weigl, Hilzingen (DE); Aarti L. Joshi, Wilmington, DE (US)

(21) Appl. No.: 15/160,238

(22) Filed: May 20, 2016

Related U.S. Application Data
(63) Continuation of application No. 15/157,927, filed on May 18, 2016, now abandoned.
(60) Provisional application No. 62/164,161, filed on May 20, 2015.

(51) Int. Cl. C07D 489/08; G01N 30/02; B01D 15/16

(52) U.S. Cl. CPC C07D 489/08; B01D 15/168; B01D 15/163; G01N 30/02

(57) ABSTRACT

The present invention is directed to a process for the preparation oxymorphone freebase, comprising hydrogenation of 14-hydroxymorphinone in DMF, to yield oxymorphone freebase, preferably oxymorphone freebase of improved appearance, purity and/or yield. The present invention is further directed to oxymorphone freebase with improved impurity profile. The present invention is further directed to an HPLC or UPLC system/method for analysis of opioid compounds.

### CROSS REFERENCE TO RELATED APPLICATIONS

This application claims the benefit of U.S. patent application Ser. No. 15/157,927, filed May 18, 2016, which also claims the benefit of U.S. Provisional Application No. 62/164,161, filed on May 20, 2015, which is incorporated by reference herein in its entirety.

### FIELD OF THE INVENTION

The present invention is directed to a process for the preparation oxymorphone freebase, comprising hydrogenation of 14-hydroxymorphinone in DMF, to yield oxymorphone freebase, preferably oxymorphone freebase of improved appearance, purity and/or yield. The present invention is further directed to oxymorphone freebase with improved impurity profile. The present invention is further directed to an HPLC or UPLC system or method for analysis of opioid compounds, including for example, determining purity and measuring the presence and amount of impurities.

### BACKGROUND OF THE INVENTION

Oxymorphone hydrochloride, also known as 4,5-epoxy-3,14-dihydroxy-17-methyl-(5α)-morphinan-6-one hydrochloride (1:1), or 14-hydroxydihydromorphinone, (C₁₇H₂₀ClNO₄, MW 337.80) is a semi-synthetic opioid analgesic. The chemical structure of oxymorphone hydrochloride is shown below

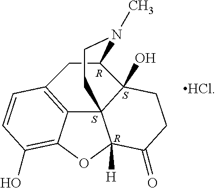

Oxymorphone HCl is indicated for the relief of moderate to severe pain. Oxymorphone HCl is also indicated as a pre-operative medication to alleviate apprehension, maintain anesthesia and as an obstetric analgesic. Additionally, oxymorphone HCl may be used to alleviate pain in patients with dyspnea associated with acute left ventricular failure and pulmonary edema.

WANG, P. X., et al., in U.S. Pat. No. 8,217,175 describe a process for the preparation of oxymorphone from oripavine, wherein the process aims to limit production of 1,1′-dimer impurities such as 2,2-bis-oxymorphone.

There remains a need for improved process(es) for the preparation of oxymorphone hydrochloride, and intermediates in the synthesis of oxymorphone hydrochloride, including but not limited to oxymorphone freebase, 14-hydroxymorphinone, etc.

### SUMMARY OF THE INVENTION

The present invention is directed to a process for the preparation of oxymorphone freebase comprising

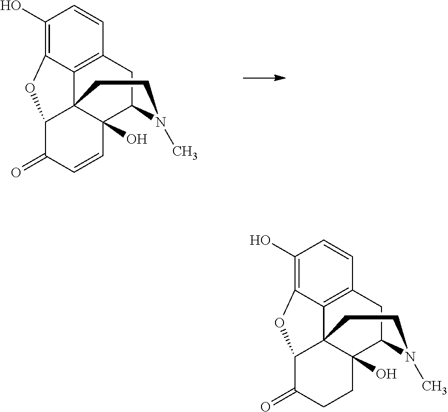

reacting 14-hydroxymorphinone with a hydrogenating agent (preferably hydrogen gas in the presence of Pd/C); optionally in the presence of ethylenediaminetetracetic acid disodium salt; optionally in the presence of a proton source (preferably in the presence of a proton source, more preferably dibasic potassium phosphate); in dimethylformamide (DMF); to yield oxymorphone as a freebase.

In an embodiment, the present invention is directed to a process for the preparation of oxymorphone freebase comprising the steps of

(a) charging 14-hydroxymorphinone, dibasic potassium phosphate, DMF and 5% Pd/C and optionally EDTA, to a nitrogen purged hydrogenation vessel;

(b) hydrogenating the 14-hydroxymorphinone with H₂ gas at 35 psi and 35° C.; to yield a reaction mixture comprising oxymorphone freebase;

(c) filtering the reaction mixture to remove the Pd/C catalyst; and yield a filtrate comprising oxymorphone freebase;

(d) adding water to the filtrate; to yield oxymorphone freebase as a precipitate; and

(e) isolating the oxymorphone freebase as a solid.

In another embodiment, the present invention is directed to a process for the preparation of oxymorphone freebase comprising the steps of

(a) charging 14-hydroxymorphinone, dibasic potassium phosphate, DMF and 5% Pd/C and optionally EDTA, to a nitrogen purged hydrogenation vessel; wherein the dibasic potassium phosphate is added in an amount of about 0.01 kg/kg relative to the amount of 14-hydroxymorphinone; wherein the DMF is added in an amount of about 3 L/kg relative to the amount of 14-hydroxymorphinone; and wherein the 5% Pd/C is added in an amount of about 0.018 kg/kg relative to the amount of 14-hydroxymorphinone;

(b) hydrogenating the 14-hydroxymorphinone with H₂ gas at 35 psi and 35° C.; to yield a reaction mixture comprising oxymorphone freebase; wherein the hydrogenation is continued until greater than about 95% of the 14-hydroxymorphinone is consumed;

(c) filtering the reaction mixture to remove the Pd/C catalyst; and yield a filtrate comprising oxymorphone freebase;

(d) adding water to the filtrate; to yield oxymorphone freebase as a precipitate; wherein the water is added in an amount of about 10 L/kg relative to the amount 14-hydroxymorphinone; and

(e) isolating the oxymorphone freebase as a solid.

The present invention is further directed to a process for the preparation of a pharmaceutically acceptable salt of oxymorphone (preferably an HCl salt of oxymorphone), comprising

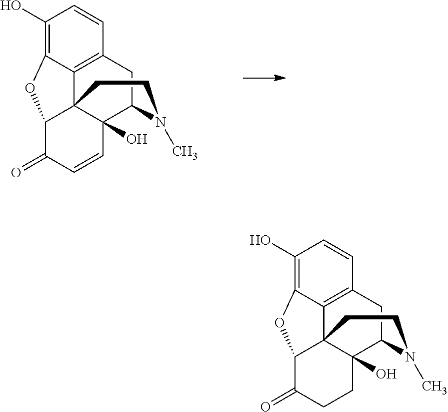

(a) reacting 14-hydroxymorphinone with a hydrogenating agent (preferably hydrogen gas in the presence of Pd/C); optionally in the presence of ethylenediaminetetracetic acid disodium salt; optionally in the presence of a proton source (preferably in the presence of a proton source, more preferably dibasic potassium phosphate); in dimethylformamide (DMF); to yield oxymorphone as a freebase; and

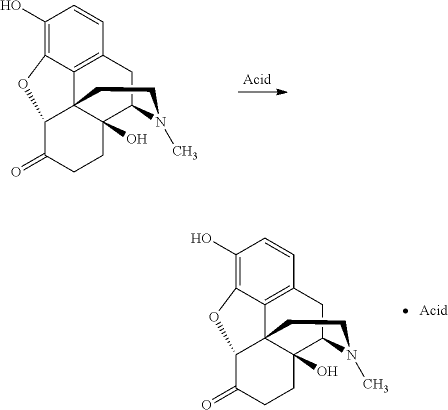

(b) reacting the oxymorphone base with an acid (preferably hydrochloric acid), to yield the corresponding oxymorphone acid addition salt.

The present invention is further directed to a process for the preparation of oxymorphone freebase, comprising

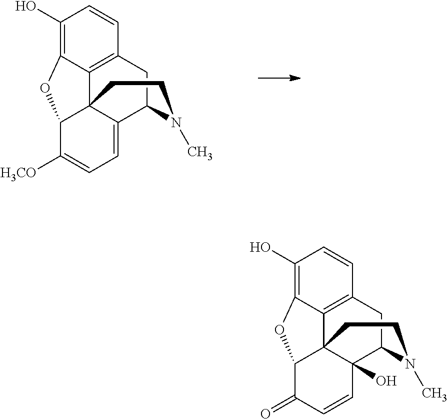

STEP 1: reacting CPS oripavine to yield 14-hydroxymorphinone; and

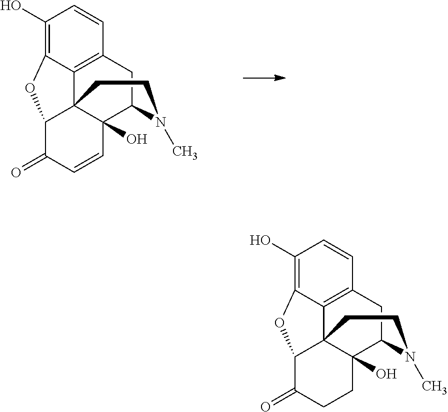

STEP 2: reacting 14-hydroxymorphinone with a hydrogenating agent (preferably hydrogen gas in the presence of Pd/C); optionally in the presence of ethylenediaminetetracetic acid disodium salt; optionally in the presence of a proton source (preferably in the presence of a proton source, more preferably dibasic potassium phosphate); in dimethylformamide (DMF); to yield oxymorphone as a freebase; and optionally,

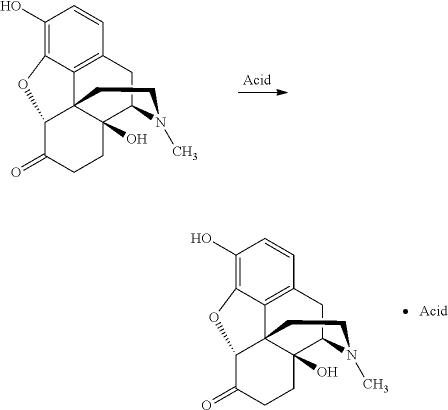

STEP 3: reacting the oxymorphone base with an acid (preferably hydrochloric acid), to yield the corresponding oxymorphone acid addition salt.

The present invention is further directed to a process for the preparation of oxymorphone freebase, comprising

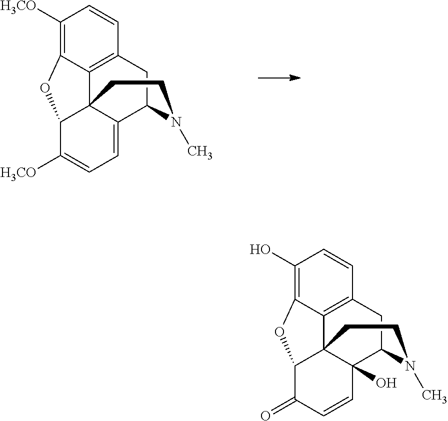

STEP 1: reacting thebaine to yield 14-hydroxymorphinone; and

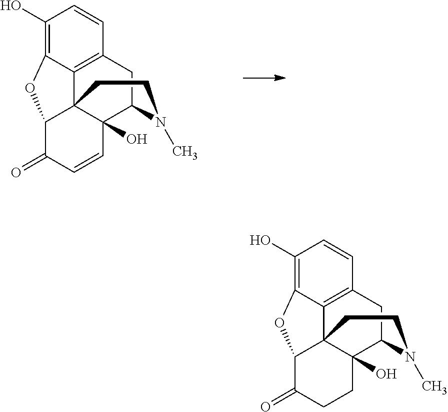

STEP 2: reacting 14-hydroxymorphinone with a hydrogenating agent (preferably hydrogen gas in the presence of Pd/C); optionally in the presence of ethylenediaminetetracetic acid disodium salt; optionally in the presence of a proton source (preferably in the presence of a proton source, more preferably dibasic potassium phosphate); in dimethylformamide (DMF); to yield oxymorphone as a freebase; and optionally

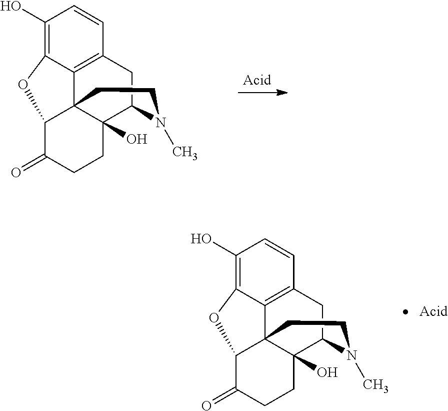

STEP 3: reacting the oxymorphone base with an acid (preferably hydrochloric acid), to yield the corresponding oxymorphone acid addition salt.

The present invention is further directed to any process for the synthesis of oxymorphone or a pharmaceutically acceptable salt thereof or for the synthesis of an intermediate useful in the synthesis of oxymorphone or a pharmaceutically acceptable salt thereof, as described herein.

The present invention is further directed to a product prepared according to any of the processes described herein. In certain embodiments, the present invention is directed to oxymorphone freebase or oxymorphone hydrochloride of improved impurity profile (improved purity) as described hereinafter.

The present invention is further directed to HPLC and UPLC system(s) and method(s) for analyzing an opioid compound (including, but not limited to natural opioid compounds, synthetic opioid compounds, opioid derivatives, etc.) or pharmaceutically acceptable salts thereof. In certain embodiments, the HPLC and UPLC methods are used to determine the purity of the opioid compound. In certain embodiments, the HPLC or UPLC method(s) are used to determine the impurity profile of an opioid compound or derivative thereof. In certain embodiments, the HPLC or UPLC system(s)/method(s) are used to determine the impurity profile of opioid compounds including, but are not limited to oxymorphone, oxycodone, hydrocodone, hydromorphone, buprenorphine, morphine, codeine, benzhydrocodone and pharmaceutically acceptable salts thereof (preferably oxymorphone, oxycodone, hydrocodone, benzyhydrocodone and pharmaceutically acceptable salts thereof). In certain embodiments, the HPLC or UPLC system(s)/method(s) for analyzing an opioid compound are as described in Example 7, Example 8, Example 9 or Example 10, which follow hereinafter.

In certain embodiments, the present invention is directed to an HPLC or UPLC system for analyzing the purity or impurity profile of an opioid compound comprising

(a) an HPLC or UPLC column for receiving mobile phases;

(b) a Mobile Phase A and a Mobile Phase B, wherein the Mobile Phase A and/or Mobile Phase B comprises a buffer and further wherein at least one of the mobile phases has (or is buffered using conventional buffers [including buffers described herein] to have) a pH equal to or within 0.5 units of the pK of the opioid compound to be analyzed.

In certain embodiments, the HPLC or UPLC system further comprises a detector capable of emitting a wavelength suitable for detecting compounds (e.g., the opioid compounds) undergoing analysis.

In certain embodiments, the present invention is directed to HPLC and UPLC system for analyzing an opioid compound (preferably analyzing the purity and/or impurity profile of an opioid compound) comprising

(a) a Waters Acquity UPLC BEH C18, 100×2.1 mm, 1.7 μm column, a Water XBridge HPLC C18, 150×3.0 mm, 3.5 μm column, a Waters XBridge Shield RP18, 150×3.0 mm, 3.5 μm column or equivalent;

(b) a column temperature in the range of from about 40° C. to about 75° C. (for example 40° C., 68° C. or 75° C.);

(c) a detector suitable for emitting a detection wavelength, preferably in the range of from about 280 nm to about 285 nm (for example at 280 nm or about 284 nm);

(d) a flow rate adjuster suitable for adjusting the flow rate of the mobile phases and run time of the analysis, preferably in the range of from about about 0.65 mL/min to about 0.70 mL/min (for example about 0.60 mL/min or about 0.65 mL/min) and the run time in the range of from about 5 min to about 1 hour, preferably a run time in the range of from about 5 min to about 30 min (for example about 9 min, about 10 min or about 30 min);

(e) a Mobile Phase A and a Mobile Phase B; wherein Mobile Phase A is a mixed phosphate buffer in water or an ammonium formate buffer in water; preferably wherein the pH of the mixed phosphate buffer in water has a pH ranging from about pH8 to about pH9, preferably, in the range of from about pH 8.2 to about pH 8.6, for example, in the range of from about pH 8.3 to about pH8.4; and/or preferably wherein the pH of the ammonium formate buffer in water has a pH ranging from about pH 9 to about pH 10, more preferably in the range of about pH 9.4 to about pH 9.8, for example a pH of about 9.6; and wherein Mobile Phase B is acetonitrile, and further wherein at least one of the mobile phases has (or is buffered to have) a pH equal to or within 0.5 units of the pK of the opioid compound to be analyzed.

In certain embodiments, the pH of at least one of the mobile phase is (or is buffered to be) less than the pK of the opioid compound to be analyzed by no more than 0.5 units

In certain embodiments, the present invention is directed to HPLC and UPLC method(s) for analyzing an opioid compound (preferably analyzing the purity and/or impurity profile of an opioid compound) comprising the steps of:

(a) selecting a Mobile Phase A and a Mobile Phase B; wherein Mobile Phase A is a mixed phosphate buffer in water or an ammonium formate buffer in water; preferably wherein the pH of the mixed phosphate buffer in water has a pH ranging from about pH 8 to about pH 9, preferably, in the range of from about pH 8.2 to about pH 8.6, for example, in the range of from about pH8.3 to about pH 8.4; and/or preferably wherein the pH of the ammonium formate buffer in water has a pH ranging from about pH 9 to about pH 10, more preferably in the range of about pH 9.4 to about pH 9.8, for example a pH of about 9.6; and wherein Mobile Phase B is acetonitrile; and preferably wherein the pH of Mobile Phase A is (or buffered to be) is equal to or within 0.5 units of the pKa of the opioid compound; and

(b) applying Mobile Phase A and Mobile Phase B with a gradient timetable selected to achieve separation of the opioid compound from any impurities present in the sample.

In certain embodiments, the present invention relates to an HPLC or UPLC method for analyzing the purity or impurity profile of an opioid compound comprising the steps of:
(a) injecting a sample of an opioid compound into an HPLC or UPLC column;
(b) applying a Mobile Phase A and a Mobile Phase B to the column; wherein Mobile Phase A is a mixed phosphate buffer in water or an ammonium formate buffer in water; wherein the pH of the mixed phosphate buffer in water is in the range of from about pH 8 to about pH 9; and wherein the pH of the ammonium formate buffer in water is preferably in the range of from about pH 9 to about pH 10; and wherein Mobile Phase B is acetonitrile; and further wherein the Mobile Phase A and the Mobile Phase B are applied with a Mobile Phase gradient selected to separate the opioid compound peaks from the impurity peaks.

In certain embodiments, the present invention is directed to HPLC and UPLC method(s) for analyzing an opioid compound (preferably analyzing the purity and/or impurity profile of an opioid compound) comprising the steps of:

(a) installing a Waters Acquity UPLC BEH C18, 100×2.1 mm, 1.7 μm column, a Water XBridge HPLC C18, 150×3.0 mm, 3.5 μm column, a Waters XBridge Shield RP18, 150×3.0 mm, 3.5 μm column or equivalent column into an HPLC or UPLC system;

(b) adjusting the column temperature to a temperature in the range of from about 40° C. to about 75° C. (for example 40° C., 68° C. or 75° C.);

(c) setting a detection wavelength of 280 nm or 285 nm;

(d) setting a flow rate for the HPLC or UPLC in the range of from about 0.65 mL/min to about 0.70 mL/min (for example about 0.60 mL/min or about 0.65 mL/min); and setting a run time in the range of from about 5 min to about 1 hour, preferably a run time in the range of from about 5 min to about 30 min (for example about 9 min, about 10 min or about 30 min);

(e) injecting a sample of the opioid compound; neat or in a suitably selected solvent;

(e) applying a Mobile Phase A and a Mobile Phase B; wherein Mobile Phase A is a mixed phosphate buffer in water or an ammonium formate buffer in water; preferably wherein the pH of the mixed phosphate buffer in water has a pH ranging from about pH8 to about pH 9, preferably, in the range of from about pH 8.2 to about pH 8.6, for example, in the range of from about pH 8.3 to about pH 8.4; and/or preferably wherein the pH of the ammonium formate buffer in water has a pH ranging from about pH 9 to about pH 10, more preferably in the range of about pH 9.4 to about pH 9.8, for example a pH of about 9.6; and wherein Mobile Phase B is acetonitrile; and wherein the pH of Mobile Phase A is (or buffered to be) equal to or within 0.5 units of the pKa of the opioid compound;

(f) applying Mobile Phase A and Mobile Phase B with a gradient timetable selected to achieve separation of the opioid compound from any impurities present in the sample; and

(g) detecting compound and impurity peaks.

In certain embodiments, the Mobile Phase gradient of the above methods is selected from the group of Gradient Timetables consisting of

Gradient Timetable A

Gradient Timetable A

Mobile Phase

| Time (min.) | % A | % B | Curve |
|---|---|---|---|
| Initial | 95 | 5 | Initial |
| 1.00 | 84 | 16 | 6 |
| 2.00 | 84 | 16 | 6 |
| 4.00 | 70 | 30 | 9 |
| 6.00 | 65 | 35 | 9 |
| 8.50 | 45 | 55 | 3 |
| 10.00 | 95 | 5 | 1 |

wherein Gradient Table A is preferred for oxymorphone and pharmaceutically acceptable salts thereof (for example HCl salts);

Gradient Table B:

Gradient Timetable B

Mobile Phase

| Time (min.) | % A | % B | Gradient Change |
|---|---|---|---|
| 0.00 | 96 | 4 | N/A |
| 1.00 | 86 | 14 | Linear |
| 3.00 | 76 | 24 | Linear |
| 8.00 | 70 | 30 | Linear |
| 12.00 | 70 | 30 | Linear |
| 18.00 | 60 | 40 | Linear |
| 21.00 | 40 | 60 | Linear |
| 26.00 | 40 | 60 | Linear |
| 26.10 | 96 | 4 | Step @26 minutes |
| 30.00 | 96 | 4 | Linear |

wherein Gradient Table B is preferred for use in HPLC measurements of oxycodone, hydrocodone, benzhydrocodone and pharmaceutically acceptable salts thereof (for example HCl salts);

Gradient Table C:

Gradient Timetable C

Mobile
Phase

| Time (min.) | % A | % B | Gradient Change |
|---|---|---|---|
| Initial | 96 | 4 | N/A |
| 0.30 | 86 | 14 | Linear |
| 0.85 | 76 | 24 | Linear |
| 2.40 | 70 | 30 | Linear |
| 3.70 | 70 | 30 | Linear |
| 5.00 | 60 | 40 | Linear |
| 6.50 | 40 | 60 | Linear |
| 7.50 | 40 | 60 | Linear |
| 9.00 | 96 | 4 | Step @ 7.5 min |

wherein Gradient Table C is preferred for use in UPLC measurements of oxycodone, oxymorphone, hydrocodone, hydromorphone and pharmaceutically acceptable salts thereof (for example HCl salts); and

Gradient Table D:

Gradient Table D

Mobile
Phase

| Time (min.) | % A | % B | Gradient Change |
|---|---|---|---|
| 0.0 | 90 | 10 | N/A |
| 10.0 | 70 | 30 | Linear |
| 25.0 | 45 | 55 | Linear |
| 25.1 | 90 | 10 | Linear |
| 30.0 | 90 | 10 | Linear |

wherein Gradient Table D is preferred for hydrocodone and pharmaceutically acceptable salts thereof (for example the bitartrate salt).

### DETAILED DESCRIPTION OF THE INVENTION

The present invention is directed to process(es) for the preparation of oxymorphone freebase comprising reacting 14-hydroxymorphinone with a suitably selected hydrogenating agent (preferably hydrogen gas in the presence of Pd/C); in the presence of ethylenediaminetetracetic acid disodium salt; optionally in the presence of a proton source (preferably in the presence of a proton source, preferably in the presence of dibasic potassium phosphate); in dimethylformamide (DMF); as described in more detail hereinafter.

The present invention is further directed to process(es) for the preparation of oxymorphone freebase from CPS oripavine or thebaine (preferably CPS oripavine), as described in more detail hereinafter.

The present invention is further directed to process(es) for the preparation of oxymorphone hydrochloride, as described in more detail hereinafter.

The present invention is further directed to process(es) for the preparation of 14-hydroxymorphinone, as described in more detail hereinafter.

The present invention is further directed to HPLC and UPLC system(s)/method(s) for analyzing an opioid compound (including, but not limited to natural opioid compounds, synthetic opioid compounds, opioid derivatives, etc.) or pharmaceutically acceptable salts thereof. In certain embodiments, the HPLC and UPLC systems/methods are used to determine the purity of the opioid compound. In certain embodiments, the HPLC or UPLC system(s)/method(s) are used to determine the impurity profile of an opioid compound or derivative thereof. In certain embodiments, the HPLC or UPLC system(s)/method(s) are used to determine the impurity profile of opioid compounds selected from the group consisting of oxymorphone, oxycodone, hydrocodone, hydromorphone, buprenorphine, morphine, codeine, benzhydrocodone and pharmaceutically acceptable salts thereof. In certain embodiments, the HPLC or UPLC system(s)/method(s) are used to determine the impurity profile of opioid compounds selected from the group consisting of oxymorphone, oxymorphone HCl, oxycodone, oxycodone HCl, hydrocodone, hydrocodone bitartrate and benzhydrocodone.

In certain embodiments, select mobile phases are eluted through an HPLC or UPLC column at an applied Mobile Phase gradient selected to achieve separation between the peaks of the opioid compound and any synthesis impurities.

In the HPLC and UPLC system(s)/method(s) of the present invention, at least one mobile phase is eluted through the HPLC or UPLC column and, preferably, at least one mobile phase is selected to be a buffer (or comprise a buffer); wherein the pH of that at least one of the mobile phase is equal to or within 0.5 units of the pK of the opioid compound (which is to be analyzed).

In certain embodiments, in the HPLC and UPLC system(s)/method(s) of the present invention, at least one mobile phase is a mixed phosphate buffer in water (preferably deionized water); wherein the pH of the mixed phosphate buffer is preferably in the range of from about pH 8 to about pH 9, preferably in the range of from about pH 8.2 to about pH 8.6, more preferably in the range of from about pH 8.3 to about pH 8.4; or an ammonium formate buffer in water; wherein the pH of the ammonium formate buffer is preferably in the range of from about pH 9 to about pH 10, more preferably in the range of from about pH 9.4 to about pH 9.8, more preferably pH 9.6.

One skilled in the art will recognize that the use of a mobile phase whose pH is close to the pK of the molecules of interest (i.e. the opioid compound(s) and/or its synthesis impurities) is contrary to conventional wisdom for HPLC/UPLC separations. Selection of the mobile phase buffer pH for acidic or basic compounds is typically been based on the pK of the substances involved. A long standing “rule” for buffer pH selection is that the pH of the mobile phase should be at least 2 pH units away from the pK of the substance(s) being analyzed, to ensure that the molecules are fully converted to their conjugate base or acid forms, to provide the most stable retention behavior of the compounds. Using this rule, the two acid/base forms would be expected to have significantly differing interactions with the mobile and stationary phases, and as a result, the retention times should be sufficiently different to effect separation and analysis.

In the present invention, for HPLC or UPLC analysis of opioid compounds and their synthesis impurities, it was unexpectedly discovered that use of at least one mobile phase having (or buffered to have, a pH close to the pK of the opioid compound (e.g., a pH of the mobile phase [or buffered mobile phase] differing from the pK of the opioid compound by plus or minus 0.5 units), was found to result in excellent separation. Although not intended to be definitive as to the reasons, it is theorized that the methods of the present invention take advantage of very small differences in pK values for very similar structures (i.e. the opioid and impurity compounds); in addition to the existing differences in the hydrophobicity of functional groups (such as ketone groups versus hydroxyl groups) on said structures.

In certain embodiments, the present invention is directed to process(es) for the preparation of oxymorphone freebase or oxymorphone hydrochloride, wherein the product is isolated as a solid, and wherein the wt % of impurities (for example organic impurities or inorganic impurities or both), as measured by an HPLC or UPLC method that sufficiently separates the opioid compound from impurities, preferably the HPLC or UPLC system(s)/method(s) described herein, is less than about 10 wt % (preferably less than about 5 wt %, more preferably less than about 2 wt %, more preferably less than about 1 wt %, more preferably less than about 0.075 wt %, more preferably less than about 0.05 wt %, more preferably less than about 0.04%, more preferably less than about 0.03%, more preferably less than about 0.02%, more preferably less than about 0.01%). In certain embodiments, the wt % of impurities is below the ICH Reporting Threshold.

In certain embodiments, the present invention is directed to process(es) for the preparation of oxymorphone freebase or oxymorphone hydrochloride, wherein the appearance/color of the isolated solid is white, off white or slight yellow.

The present invention is further directed to a product prepared according to any of the processes as described herein. In certain embodiments, the present invention is directed to a product prepared according to any of the processes described herein, wherein the wt % of impurities in the product, as measured by an HPLC or UPLC method that sufficiently separates the opioid compound from impurities, preferably the HPLC or UPLC system(s)/method(s) described herein, is less than about 10 wt %, preferably less than about 5 wt %, more preferably less than about 2 wt %, more preferably less than about 1 wt %, more preferably less than about 0.075 wt %, more preferably less than about 0.05 wt %%, more preferably less than about 0.04%, more preferably less than about 0.03%, more preferably less than about 0.02%, more preferably less than about 0.01%.

In certain embodiments, the present invention is directed to oxymorphone freebase (including, but not limited to oxymorphone freebase prepared according to any of the process(es) described herein) or oxymorphone hydrochloride (including, but not limited to oxymorphone hydrochloride prepared according to any of the process(es) described herein); wherein the purity of the oxymorphone freebase or oxymorphone hydrochloride, as measured by an HPLC or UPLC method that sufficiently separates the opioid compound from impurities, preferably the HPLC or UPLC system(s)/method(s) described herein, is in the range of from about 95% to about 100%, or any amount or range therein, preferably, the purity is at least about 97.5%, more preferably at least about 98%, preferably at least 98.1%, more preferably at least 98.2%, more preferably at least 98.3%, more preferably at least 98.4%, more preferably at least 98.5%, more preferably at least 98.6%, more preferably at least 98.7%, more preferably at least 98.8%, more preferably at least 98.9%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, more preferably at least 99.9%.

In certain embodiments, the present invention is directed to a product prepared according to any of the processes described herein (preferably, oxymorphone freebase or oxymorphone hydrochloride), wherein the individual wt % of one or more (preferably one to two, more preferably one to four) of the following impurities (a) 14-hydroxymorphinone, (b) 14-hydroxy-dihydromorphine (α), (c) 8-hydroxyoxymorphone, (d) hydromorphone and/or 2,2-bis-oxymorphone is, as measured by an HPLC or UPLC method that sufficiently separates the opioid compound from such impurities, preferably the HPLC or UPLC system(s)/method(s) described herein, individually less than or equal to about 0.5 wt %, preferably less than about 0.3 wt %, more preferably less than about 0.2 wt %, more preferably less than about 0.1 wt %, more preferably less than about 0.05 wt %, most preferably in an amount below the ICH Reporting Threshold (for example in the range of from about 0.25 wt % to about 0.05 wt % or in a range of from about 0.1 wt % to about 0.05 wt %).

In certain embodiments, the present invention is directed to a product prepared according to any of the processes described herein (preferably, oxymorphone freebase or oxymorphone hydrochloride), wherein the sum total wt % of one or more (preferably one to two, more preferably one to four) of the following impurities (a) 14-hydroxymorphinone, (b) 14-hydroxy-dihydromorphine (α), (c) 8-hydroxyoxymorphone, (d) hydromorphone, and/or 2,2-bis-oxymorphone is, as measured by an HPLC or UPLC method that sufficiently separates the opioid compound from such impurities, preferably the HPLC or UPLC system(s)/method(s) described herein, less than or equal to about 2.0 wt %, preferably less than about 1.5 wt %, more preferably less than about 1.0 wt %, more preferably less than about 0.5 wt %, more preferably less than about 0.25 wt % more preferably less than about 0.1 wt % (for example, in a range of from about 1.0 wt % to about 0.1 wt %, in a range of from about 0.5 wt % to about 0.1 wt %, or in a range of from about 0.5 wt % to about 0.2 wt %).

In certain embodiments, the present invention is directed to a product prepared according to any of the processes described herein (preferably, oxymorphone freebase or oxymorphone hydrochloride), wherein the individual wt % of one or more (preferably one to two, more preferably one to four, more preferably one to six, more preferably one to eight) of the following impurities (a) oxymorphone-N-oxide, (b) 8-hydroxyoxymorphone, (c) hydromorphone, (d) 14-hydroxymorphinone, (e) 14-hydroxydihydromorphine (α), (f) oxycodone, (g) 2,2-bis-oxymorphone, and/or (h) any other individual unspecified or unidentified impurity is, as measured by an HPLC or UPLC method that sufficiently separates the opioid compound from such impurities, preferably the HPLC or UPLC system(s)/method(s) described herein, individually less than or equal to about 0.5 wt %, preferably less than about 0.3 wt %, more preferably less than about 0.2 wt %, more preferably less than about 0.1 wt %, more preferably less than about 0.05 wt %, most preferably in an amount below the ICH Reporting Threshold (for example in the range of from about 0.25 wt % to about 0.05 wt % or in a range of from about 0.1 wt % to about 0.05 wt %).

In certain embodiments, the present invention is directed to a product prepared according to any of the processes described herein (preferably, oxymorphone freebase or oxymorphone hydrochloride), wherein the sum total wt % of one or more (preferably one to two, more preferably one to four, more preferably one to six, more preferably one to eight) of the following impurities (a) oxymorphone-N-oxide, (b) 8-hydroxyoxymorphone, (c) hydromorphone, (d) 14-hydroxymorphinone, (e) 14-hydroxydihydromorphine (α), (f) oxycodone, (g) 2,2-bis-oxymorphone, and/or (h) any other individual unspecified or unidentified impurity is, as measured by an HPLC or UPLC method that sufficiently separates the opioid compound from such impurities, preferably the HPLC or UPLC system(s)/method(s) described herein, less than or equal to about 2.0 wt %, preferably less than about 1.5 wt %, more preferably less than about 1.0 wt %, more preferably less than about 0.5 wt %, more preferably less than about 0.25 wt % more preferably less than about 0.1 wt % (for example, in a range of from about 1.0 wt % to about 0.1 wt %, in a range of from about 0.5 wt % to about 0.1 wt %, or in a range of from about 0.5 wt % to about 0.2 wt %).

The process(es) of the present invention provide a means of preparing oxymorphone freebase or oxymorphone hydrochloride at higher throughput (faster reaction time/faster cycle time), reduced cost and/or with higher product quality, as determined by for example color/appearance, % impurities, % organic impurities, % inorganic impurities, etc.

In an embodiment, the present invention is directed to process(es) for the preparation of oxymorphone or pharmaceutically acceptable salts thereof, wherein the hydrogenation of 14-hydroxymorphinone in DMF (dimethylformamide) provides a means of removing (and purging) impurities from the oxymorphone product. In another embodiment, the present invention is directed to process(es) for the preparation of oxymorphone or pharmaceutically acceptable salts thereof wherein the process(es) minimize (or produce low amounts) of inorganic salt impurities.

As used herein, unless otherwise noted, the term “opioid compound”, when referring to compounds which may be analyzed using the HPLC and/or UPLC systems/methods disclosed herein, include natural opioids, synthetic opioids and opioid derivatives. In certain embodiments, the opioid compound is selected from the group consisting of morphine, codeine, oxymorphone, oxycodone, hydrocodone, hydromorphone, buprenorphine, benzhydrocodone, and pharmaceutically acceptable salts thereof. Preferably, the opioid compound is selected from the group consisting of oxymorphone, oxycodone, hydrocodone, hydromorphone and pharmaceutically acceptable salts thereof. More preferably, the opioid compound is selected from the group consisting of oxymorphone, oxycodone and pharmaceutically acceptable salts thereof.

As used herein, the notation “*” shall denote the presence of a stereogenic center.

Where the compounds according to the present invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

Furthermore, it is intended that within the scope of the present invention, any element, shall comprise all isotopes and isotopic mixtures of said element, either naturally occurring or synthetically produced, either with natural abundance or in an isotopically enriched form. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. The isotopes may be radioactive or non-radioactive. Radiolabelled compounds of formula (I) may comprise a radioactive isotope selected from the group of ³H, ¹¹C, ¹⁸F, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ³H, ¹¹C and ¹⁸F.

Abbreviations used in the specification, particularly the Schemes and Examples, are as follows:
CPS Oripavine=Concentrated Poppy Straw Oripavine
DIPEA=Diisopropylethylamine
DMF=N,N-Dimethylformamide
EDTA=Ethylene Diamine Tetraacetic Acid
HPLC=High Performance Liquid Chromatography
14-HM=14-Hydroxymorphinone
14-HDHM=14-Hydroxy-dihydromorphine (α)
ICH=International Council for Harmonisation of Technical Requirements for Pharmaceuticals for Human Use
MCPBA or mCPBA=meta-Chloroperoxybenzoic Acid
NLT=Not Less Than
NMT=Not More Than
OMFB=Oxymorphone Freebase
Pd/C=Palladium on Carbon Catalyst
ROS=Residual Organic Solvent
RRT=Relative Retention Time
TEA=Triethylamine
TMA=Trimethylamine
UPLC=Ultra Performance Liquid Chromatography

As used herein, unless otherwise noted, the term “isolated form” shall mean that the compound is present in a form which is separate from any solid mixture with another compound(s), solvent system or biological environment. In certain embodiments, the present invention is directed to process(es) for the preparation of oxymorphone freebase or oxymorphone hydrochloride in an isolated form.

As used herein, unless otherwise noted, the term “substantially free of a corresponding salt form(s)” when used to described the compound of formula (I) shall mean that mole percent of the corresponding salt form(s) in the isolated base of formula (I) is less than about 5 mole percent, preferably less than about 2 mole percent, more preferably, less than about 0.5 mole percent, most preferably less than about 0.1 mole percent. In certain embodiments, the present invention is directed to process(es) for the preparation of oxymorphone freebase in a form which is substantially free of corresponding salt form(s).

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

Examples of suitable solvents, bases, reaction temperatures, and other reaction parameters and components are provided in the detailed descriptions which follow herein. One skilled in the art will recognize that the listing of said examples is not intended, and should not be construed, as limiting in any way the invention set forth in the claims which follow thereafter.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems. One skilled in the art will further recognize that wherein two consecutive reaction or process steps are run without isolation of the intermediate product (i.e. the product of the first of the two consecutive reaction or process steps), then the first and second reaction or process steps may be run in the same solvent or solvent system; or alternatively may be run in different solvents or solvent systems following solvent exchange, which may be completed according to known methods.

One skilled in the art will further recognize that the reaction or process step(s) as herein described (or claimed) are allowed to proceed for a sufficient period of time until the reaction is complete, as determined by any method known to one skilled in the art, for example, chromatography (e.g. HPLC, TLC, etc.). In this context a “completed reaction or process step” shall mean that the reaction mixture contains a significantly diminished amount of the starting material(s)/reagent(s) and a significantly increased amount of the desired product(s), as compared to the amounts of each present at the beginning of the reaction.

In an embodiment, the amount of staring material remaining in the reaction mixture upon completion of the reaction or process step is less than about 50 mole %, more preferably less than about 25 mole %, more preferably less than about 20 mole %, more preferably, less than about 15 mole %, more preferably less than about 10 mole %, more preferably less than about 5 mole %, more preferably less than about 2 mole %.

In another embodiment, the amount of any desired reaction product, present in the reaction mixture, upon completion of the reaction or process step is greater than about 50 mole %, more preferably greater than about 75 mole %, more preferably greater than about 80 mole %, more preferably, greater than about 85 mole %, more preferably greater than about 90 mole %, more preferably greater than about 95 mole %, more preferably greater than about 98 mole %.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (% ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α−obs]/[α−max])×100.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid.

Representative bases which may be used in the preparation of pharmaceutically acceptable salts include, but are not limited to, the following: bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The present invention is directed to a process for the preparation of oxymorphone freebase comprising hydrogenation of 14-hydroxymorphinone in DMF, as described in more details in Scheme 1, below.

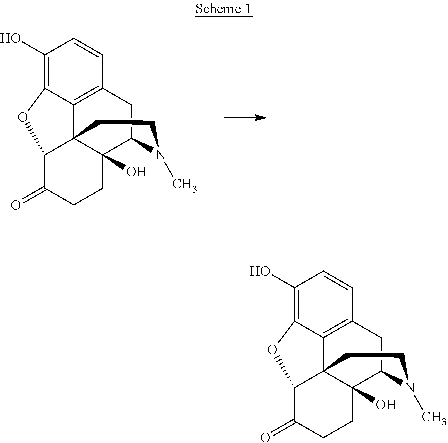

Accordingly, 14-hydroxymorphinone is dissolved in DMF (dimethylformamide) and then reacted with a suitably selected hydrogenating agent, preferably hydrogen gas; in the presence of a suitably selected catalyst such as Pd/C catalyst, and the like; in the presence of a suitably selected proton source such as dibasic potassium phosphate, and the like; and optionally in the presence of a suitably selected metal scavenger such as EDTA, and the like; to yield oxymorphone freebase.

More particularly, 14-hydroxymorphinone is dissolved in DMF; wherein the DMF is present in any amount sufficient to dissolve the 14-hydroxymorphinone; preferably, the DMF is present in an amount in the range of from about 1 L/kg to about 10 L/kg (relative to the amount (mass) of 14-hydroxymorphinone), or any amount or range therein, more preferably in an amount in the range of from about 1 L/kg to about 5 L/kg, more preferably in an amount in the range of from about 2 L/kg to about 6 L/kg, more preferably in an amount in the range of from about 2 L/kg to about 4 L/kg, more preferably in an amount in the range of from about 2.3 L/kg to about 4 L/kg, more preferably in an amount in the range of from about 2.5 L/kg to about 3.5 L/kg, more preferably in an amount of about 3 L/Kg;

and reacted with a suitably selected hydrogenating agent such as hydrogen gas, and the like; wherein the hydrogen gas is preferably present at a pressure in the range of from about 10 psi to about 75 psi, more preferably at a pressure in the range of from about 20 psi to about 50 psi, or any amount or range therein, more preferably at a pressure in the range of from about 30 psi to about 50 psi, more preferably at a pressure in the range of from about 30 psi to about 40 psi, more preferably at a pressure of about 35 psi;

in the presence of a suitably selected catalyst such as Pd/C, and the like, for example 5% Pd/C, 10% Pd/C, and the like, preferably 5% Pd/C; wherein the catalyst is preferably present in an amount in the range of from about 0.5 wt % to about 2 wt % (relative to the amount (weight) of 14-hydroxymorphinone), or any amount or range therein, more preferably present in an amount in the range of from about 1 wt % to about 2 wt %, more preferably in an amount in the range of from about 1.5 wt % to about 2 wt %, more preferably in an amount of about 1.8 wt %;

optionally, in the presence of a suitably selected proton source such as dibasic potassium phosphate, monobasic potassium phosphate, phosphoric acid, and the like, preferably dibasic potassium phosphate; wherein the proton source is preferably present in an amount in the range of from about 0.001 kg/kg to about 0.05 kg/kg (relative to the amount (weight) of 14-hydroxymorphinone), or any amount or range therein, more preferably in an amount in the range of from about 0.001 kg/kg to about 0.02 kg/kg, more preferably in an amount in the range of from about 0.001 kg/kg to about 0.015 kg/kg, more preferably in an amount of about 0.01 kg/kg;

and optionally in the present of a suitably selected metal scavenger such as a suitably selected carboxylic acid (such as EDTA, and the like), a sulfonic acid (such as propylsulfonic acid, and the like), an amine (such as tris-(2-aminoethyl)amino, and the like) or a thiol (such as 1-propanethiol, and the like), optionally on a solid support, preferably EDTA; wherein the metal scavenger is present in an amount in the range of from about 0.001 kg/kg to about 0.05 kg/kg (relative to the amount of 14-hydroxymorphinone), or any amount or range therein, more preferably in an amount in the range of from about 0.01 kg/kg to about 0.04 kg/kg, more preferably in an amount in the range of from about 0.01 kg/kg to about 0.03 kg/kg, more preferably in an amount of about 0.02 kg/kg;

preferably at a temperature greater than about room temperature, preferably at a temperature in the range of from about 20° C. to about 50° C., or any temperature or range therein, more preferably at a temperature in the range of from about 25° C. to about 45° C., more preferably at a temperature in the range of from about 30° C. to about 40° C., more preferably at a temperature of about 35° C.; to yield oxymorphone as its corresponding freebase.

In an embodiment of the present invention, the amount of DMF is sufficient to at least partially dissolve the 14-hydroxymorphinone.

One skilled in the art may theorize that the addition of a greater amount of catalyst may result in increased rate of reaction. However, for the process(es) of the present invention, it has been observed that increasing the amount of catalyst added to the reaction mixture adversely affect product color and/or appearance.

Preferably, the mixture comprising the oxymorphone freebase is filtered to remove the catalyst; and the filtercake rinsed with DMF.

Preferably, the oxymorphone freebase is isolated, for example according to known methods.

In an embodiment, to the reaction mixture comprising oxymorphone freebase (preferably, a mixture of the filtrate resulting from the removal of the catalyst combined with any DMF wash) is added sodium hydrosulfite (to improve color or appearance); wherein the sodium hydrosulfite is added in an amount in the range of from about 0.001 kg/kg to about 0.05 kg/kg (relative to the amount (weight) of 14-hydroxymorphinone), or any amount or range therein, preferably in an amount in the range of from about 0.005 kg/kg to about 0.03 kg/kg, more preferably in an amount in the range of from about 0.008 kg/kg to about 0.02 kg/kg, more preferably in an amount of about 0.01 kg/kg.

The oxymorphone freebase is then preferably precipitated from the reaction mixture by addition of water; wherein the water is added in an amount in the range of from about 6 L/kg to about 12 L/kg (relative to the amount (weight) of 14-hydroxymorphinone), or any amount or range therein, more preferably, in an amount in the range of from about 8 L/kg to about 12 L/kg, more preferably, in an amount in the range of from about 10 L/kg;

and wherein the temperature of the reaction mixture is preferably controlled to a temperature less than about 40° C. (for example, by jacket temperature, rate of addition of water, etc.), preferably the temperature of the reaction mixture is controlled to a temperature in the range of from about 10° C. to about 30° C., or any temperature or range therein, more preferably, the temperature of the reaction mixture is controlled to a temperature in the range of from about 20° C. to about 35° C., more preferably, more preferably the temperature of the reaction mixture is controlled to a temperature in the range of from about 30° C. to about 35° C.

It has been observed that addition of the water to the reaction mixture results in an initial exotherm, which raises the temperature of the reaction mixture. Preferably, the temperature of the reaction mixture is allowed to increase with the generated exotherm. More preferably, the temperature of the reaction mixture is allowed to increase to a temperature of less than about 50° C., preferably to a temperature less than about 40° C., preferably to a temperature in the range of from about 30° C. to about 35° C. The oxymorphone freebase is then preferably isolated, for example, by further cooling the reaction mixture to a temperature less than about 35° C., preferably to a temperature in the range of from about 10° C. to about 30° C., or any temperature or range therein, more preferably to a temperature in the range of from about 15° C. to about 25° C., more preferably to a temperature of about 20° C., and filtered; followed by optional washing of the filtercake with a suitably selected solvent such as water. Preferably, the filtercake (comprising the isolated oxymorphone freebase) is dried, optionally under a temperature greater than room temperature and/or under reduced pressure.

Although not intended to be limiting or definitive, it is theorized that hydrogenation of 14-hydroxymorphinone under basic conditions (as in the process(es) of the present invention), results in a decrease in the formation of 14-hydroxydihydromorphine (α) (14-HDHM), 8-hydroxyoxymorphone (which on further conversion in subsequent reaction steps of the present invention as herein described, leads to lower amounts of the 14-hydroxymorphinone impurity), and 2,2′-bis-oxymorphone, each of which is an undesired by-product; thereby increasing product purity and yield.

In an embodiment, the present invention is directed to a product (preferably oxymorphone freebases or oxymorphone hydrochloride) prepared according to any of the processes described herein, wherein the amount (e.g. wt %) of 2,2′-bis-oxymorphone impurity present in the product is, as measured by an HPLC or UPLC method that sufficiently separates the opioid compound from impurities, preferably the HPLC or UPLC system(s)/method(s) described herein, less than or equal to about 0.5 wt %, preferably less than about 0.3 wt %, more preferably less than about 0.2 wt %, more preferably less than about 0.1 wt %, more preferably less than about 0.05 wt %.

In an embodiment, the present invention is directed to a product (preferably oxymorphone freebases or oxymorphone hydrochloride) prepared according to any of the processes described herein, wherein the amount (e.g. wt %) of 14-hydroxy-dihydromorphine (α) impurity present in the product is, as measured by an HPLC or UPLC method that sufficiently separates the opioid compound from impurities, preferably the HPLC or UPLC system(s)/method(s) described herein, less than or equal to about 0.5 wt %, preferably less than about 0.3 wt %, more preferably less than about 0.2 wt %, more preferably less than about 0.1 wt %, more preferably less than about 0.05 wt %.

In an embodiment, the present invention is directed to a product (preferably oxymorphone freebases or oxymorphone hydrochloride) prepared according to any of the processes described herein, wherein the amount (e.g. wt %) of 8-hydroxyoxymorphone impurity present in the product is, as measured by an HPLC or UPLC method that sufficiently separates the opioid compound from impurities, preferably the HPLC or UPLC system(s)/method(s) described herein, less than or equal to about 0.5 wt %, preferably less than about 0.3 wt %, more preferably less than about 0.2 wt %, more preferably less than about 0.1 wt %, more preferably less than about 0.05 wt %.

Additionally, in the process(es) of the present invention, the hydrogenation of 14-hydroxymorphinone is carried out in DMF and the oxymorphone freebase is precipitated from water, without the need for additional acid or base, resulting in a decrease in the formation of inorganic salt impurities (which may require additional processing to remove).

Additionally, in the process(es) of the present invention, the hydrogenation of 14-hydroxymorphinone is carried out in the presence of a proton source, preferably dibasic potassium phosphate, resulting in a decrease in the color of the isolated oxymorphone freebase (for example from a grey to an off-white color).

One skilled in the art will recognize that in the process of the present invention as described in Scheme 1 above, 14-hydroxymorphinone may be prepared according to known methods, for example as described in more detail in Scheme 2, which follows hereinafter. Alternatively, 14-hydroxymorphinone may be prepared from thebaine, according to for example, the process as described in Example 4, which follows hereinafter.

The present invention is further directed to a two-step process for the preparation of oxymorphone freebase, as described in more detail in Scheme 2 below.

### Step 1: Oxidation of CPS Oripavine to 14-Hydroxymorphinone

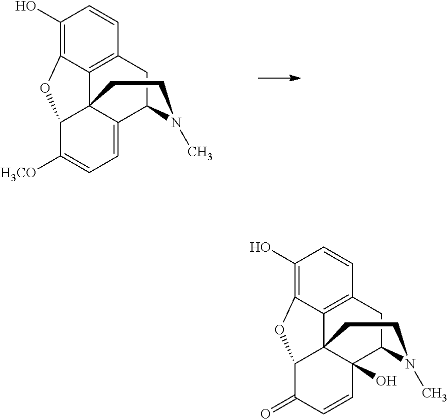

### Step 2: Hydrogenation of 14-Hydroxymorphinone to Oxymorphone Freebase

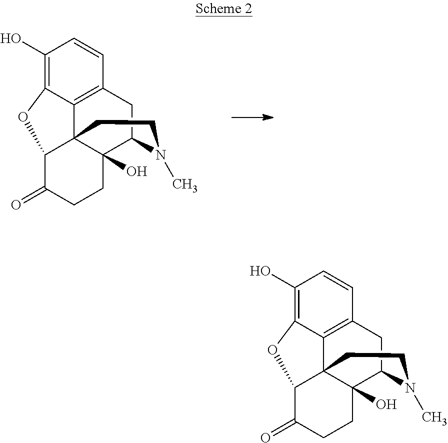

Accordingly, in the first step, concentrated poppy straw (CPS) oripavine (also known as 6,7,8,14-tetradehydro-4,5α-epoxy-6-methoxy-17-methylmorphinan-3-ol) is oxidized to yield 14-hydroxymorphinone.

In an embodiment, CPS oripavine is dissolved in a mixture of water and a suitably selected peroxyacid forming agent (an agent which, when mixed with hydrogen peroxide will form the corresponding peroxyacid in situ), such as a suitably selected carboxylic acid, a suitably selected carboxylic anhydride or a suitably selected acyl halide, preferably a suitably selected carboxylic acid, as formic acid, acetic acid, propionic acid, and the like, more preferably formic acid, more preferably 90% to 98% formic acid, more preferably 90% formic acid or 98% formic acid;

wherein the water (preferably the total amount of water present during the reaction) is present in an amount in the range of from about 0.1 kg/kg to about 5 kg/kg (relative to the amount (weight) of CPS oripavine), or any amount or range therein, preferably, in an amount in the range of from about 0.25 kg/kg to about 3 kg/kg, more preferably, in an amount in the range of from about 0.5 kg/kg to about 2 kg/kg, more preferably in an amount in the range of from about 0.8 kg/kg to about 2 kg/kg, more preferably in an amount in the range of from about 1 kg/kg to about 2 kg/kg, more preferably in an amount in the range of from about 1.4 kg/kg to about 1.8 kg/kg, more preferably in an amount of about 1.6 kg/kg; (in an example, the amount of water present in the reaction is in the range of from about 1.4 L/kg to about 1.6 L/kg relative to the amount of CPS oripavine);

wherein the peroxyacid forming agent, preferably the carboxylic acid (preferably 90% formic acid or 98% formic acid), is present in an amount in the range of from about 0.1 kg/kg to about 2 kg/kg (relative to the amount (weight) of CPS oripavine), or any amount or range therein, preferably, in an amount in the range of from about 0.25 kg/kg to about 1.8 kg/kg, more preferably, in an amount in the range of from about 0.5 kg/kg to about 1.5 kg/kg, more preferably in an amount in the range of from about 0.75 kg/kg to about 1.25 kg/kg, more preferably in an amount in the range of from about 0.9 kg/kg to about 1.2 kg/kg, more preferably in an amount in the range of from about 1.0 kg/kg to about 1.1 kg/kg, more preferably in the amount of about 1.04 kg/kg; (in an example, the peroxyacid forming agent is a 90% formic acid, present in an amount in the range of from about 1.1 kg/kg to about 1.25 kg/kg (preferably, about 1.13 kg/kg 0.95 L/kg) relative to the amount of CPS oripavine);

and wherein the weight/weight ratio of water to peroxyacid forming agent (preferably carboxylic acid) is preferably in a range of from about 2:1 to about 1:1, more preferably in a range of from about 1.8:1 to about 1.4:1, more preferably in a ratio of about 1.6:1.

One skilled in the art will recognize that the total amount of water used in the reaction step described above shall include the water added to dissolve the CPS oripavine and any water which is present in the peroxyacid forming agent (preferably the carboxylic acid). For example, if the peroxyacid forming agent is formic acid and the formic acid is added as 90% formic acid, the total amount of water shall include the amount of water present in the formic acid plus the amount of water added directly to dissolve the CPS oripavine.

The resulting mixture is preferably stirred at a temperature in the range of from about room temperature to about 40° C., preferably at a temperature in the range of from about 30° C. to about 40° C., and optionally filtered to remove any insoluble matter. The resulting filtrate is then preferably cooled to about 20° C. (or to room temperature) prior to the next addition.

In an embodiment of the present invention, to the filtrate is then optionally added, a suitably selected metal scavenger such as a suitably selected carboxylic acid (such as EDTA, and the like), a sulfonic acid (such as propyl sulfonic acid, and the like), an amine (such as tris-(2-aminoethyl)amine, and the like) or a thiol (such as 1-propanethiol, and the like), optionally on a solid support, preferably EDTA; wherein the metal scavenger is present in an amount in the range of from about 0.001 kg/kg to about 0.05 kg/kg (relative to the amount (weight) of CPS oripavine), or any amount or range therein, preferably in an amount in the range of from about 0.01 kg/kg to about 0.03 kg/kg, more preferably in an amount in the range of from about 0.018 kg/kg to about 0.22 kg/kg, more preferably in an amount of about 0.02 kg/kg.

To the filtrate (or mixture of filtrate and metal scavenger) mixture are then added (sequentially and separately) (a) a suitably selected peroxide (preferably hydrogen peroxide) followed by (b) a suitably selected acid (preferably sulfuric acid).

Accordingly, to the filtrate (or mixture of filtrate and metal scavenger) mixture is first added a suitably selected peroxide such as hydrogen peroxide, a suitably selected peroxyacid salt (such as sodium peroxide, and the like), and the like, preferably about 30% hydrogen peroxide (for example, hydrogen peroxide in the range of about 30% to about 35%, preferably hydrogen peroxide in the range of about 30% to about 32%); wherein the peroxide is present in an amount in the range of from about 0.5 to about 3 molar equivalents (relative to the moles of CPS oripavine), or any amount or range therein, preferably in an amount in the range of about 0.75 to about 2.0 molar equivalents, more preferably in an amount in the range of about 0.9 to about 1.5 molar equivalents, more preferably, in an amount in the range of from about 1.0 to about 1.2 molar equivalents, more preferably in an amount in the range of from about 1.05 to about 1.2 molar equivalents, for example 1.14 molar equivalents; (in an example, about 30-32% wt/wt hydrogen peroxide is added in an amount of about 1.14 molar equivalents, relative to the amount of CPS oripavine);

and wherein during addition of the peroxide, the temperature of the reaction mixture is maintained in the range of from about 20° C. to about 25° C. (by for example, jacket temperature control, rate of peroxide addition, etc.);

and then added a suitably selected acid such as sulfuric acid (to catalyze the peroxyacid formation), and the like; wherein the acid is present in an amount in the range of from about 0.25 to about 0.75 molar equivalents (relative to the moles of CPS oripavine), or any amount or range therein, preferably in an amount in the range of from about 0.4 to about 0.6 molar equivalents, more preferably in an amount of about 0.5 molar equivalents; (preferably, in an embodiment, an amount in the range of from about 0.13 kg/kg to about 0.20 kg/kg (relative to the amount (weight) of CPS oripavine); (in an example, sulfuric acid is added in an amount in the range of from about 0.07 to about 0.11 L/kg, relative to the amount of CPS oripavine);

and wherein the temperature of the reaction mixture is preferably maintained below about 45° C. (for example in the range of from about 20° C. to about 45° C.), more preferably, at a temperature less than about 35° C., more preferably at a temperature less than about 30° C. (for example, at a temperature less than about 25° C.), more preferably at a temperature in the range of from about 20° C. to about 30° C., more preferably at a temperature in the range of from about 20° C. to about 28° C., more preferably at a temperature in the range of from about 20° C. to about 24° C.; to yield 14-hydroxymorphinone.

In an embodiment of the present invention, the temperature of the reaction, the amount of hydrogen peroxide, the amount of sulfuric acid and/or the amount of water are selected to maximize yield of the desired product, while minimizing the production of impurities and/or by-products.

In one embodiment of the present invention, the amount of hydrogen peroxide added to the reaction mixture is less than or equal to about 1.2 molar equivalents (relative to the moles of CPS oripavine). In another embodiment of the present invention, the amount of sulfuric acid added to the reaction mixture is less than or equal to about 0.6 molar equivalents (relative to the moles of CPS oripavine). In another embodiment of the present invention, the temperature of the reaction mixture is maintained at less than or equal to about 30° C., preferably at a temperature in the range of from about 20° C. to about 25° C., or any amount or range therein.

One skilled in the art will further recognize that the oxidation reaction of the first step of the process(es) of the present invention, as described above, may alternatively be completed using a pre-formed peroxyacid such as mCPBA, performic acid, peracetic acid, and the like; rather than forming the peroxyacid in situ, as described above. One skilled in the art will recognize that some commercially available peroxyacids (such as for example, peracetic acid) may be supplied in a form which contains small amounts of sulfuric acid.

One skilled in the art will recognize that the oxidation reaction of the first step of the process(es) of the present invention, as described above, is preferably stopped at the optimal point (e.g. at the point at which the target conversion amount or starting material consumption amount is reached), based on continuous or periodic testing (e.g. HPLC or other assay method) of the reaction mixture/product stream.

The 14-hydroxymorphinone is preferably isolated as a solid, according to known methods, for example by filtration.

In an embodiment of the present invention, the reaction mixture comprising the 14-hydroxymorphinone product is quenched by addition of a suitably selected peroxide neutralizer such as sodium sulfite, sodium bis-sulfite, and the like, preferably sodium sulfite, more preferably aqueous sodium sulfite; wherein the peroxide neutralizer is preferably present in an amount sufficient to quench any remaining peroxyacid, preferably in an amount in the range of from about 0.01 to about 0.5 molar equivalents (relative to the moles of CPS oripavine), or any amount or range therein, more preferably, in an amount in the range of from about 0.05 to about 0.25 molar equivalents, more preferably in an amount in the range of from about 0.05 to about 0.15 molar equivalents, more preferably in an amount in the range of from about 0.05 to about 0.1 molar equivalents, more preferably in an amount of about 0.08 molar equivalents (in an example, the peroxide neutralizer is added in an amount in the range of from about 0.02 to about 0.11 kilograms; in another example, the peroxide neutralizer is aqueous sodium sulfite, wherein the aqueous sodium sulfite is added as an aqueous solution of sodium sulfite in 0.28 L/kg of water relative to CPS oripavine);

and wherein the temperature of the reaction mixture is preferably maintained at a temperature less than about 30° C., more preferably, the temperature is maintained in the range of from about 15° C. to about 25° C., more preferably, the temperature is maintained in the range of from about 20° C. to about 25° C. (for example, by controlling addition rate).

In an embodiment of the present invention, the amount of peroxide neutralizer (for example sodium sulfite) added to the reaction mixture is an amount sufficient to neutralize any excess peroxide or peroxyacid present in the reaction mixture. In another embodiment of the present invention, to maximize yield and/or minimize impurity production, the amount of peroxide neutralizer (preferably sodium sulfite) is the minimum amount necessary to neutralize any peroxyacid or peroxide remaining in the reaction mixture, the amount of which may be calculated from the excess peroxide initially charged (added) to the reaction mixture. One skilled in the art will recognize that the presence or absence of residual peroxide or peroxyacid may be determined by methods know in the art, for example by testing with colorimetric strips.

To the resulting reaction mixture is then optionally added water; wherein the water is added in an amount in the range of from about 1 to about 10 kilograms (relative to one kilogram of CPS oripavine), or any amount or range therein, preferably in an amount in the range of from about 2 to about 8 kilograms, more preferably in an amount in the range of from about 3 to about 6.5 kilograms, more preferably in an amount in the range of from about 4 to about 6 kilograms, more preferably, in an amount of about 5 kilograms.

The 14-hydroxymorphinone is then preferably precipitated by addition of a suitably selected organic amine base such as TEA (triethylamine), TMA (trimethylamine), DIPEA (diisopropylethylamine), pyridine, and the like, preferably, TEA; wherein the organic amine base is added in an amount sufficient to adjust the pH of the reaction mixture to a pH sufficient to precipitate 14-hydroxymorphinone, preferably, the organic amine base is added in an amount sufficient to adjust the pH of the reaction mixture to a pH in the range of from about pH 8 to about pH 10, or any pH or pH range therein, more preferably, to a pH in the range of from about pH 8.5 to about pH 9.5, more preferably, to a pH in the range of from about pH 8.6 to about pH 9.2, more preferably to a pH of about pH 8.9;

and wherein, during addition of the organic amine base, the temperature of the reaction mixture is maintained below about 50° C. (for example, by controlling jacket temperature, the addition rate of the organic amine base, etc.), more preferably, the temperature is maintained below about 40° C., more preferably the temperature is maintained in the range of from about 10° C. to about 40° C., more preferably, the temperature is maintained in the range of from about 15° C. to about 35° C., more preferably, the temperature is maintained in the range of from about 20° C. to about 32° C., more preferably the temperature in maintained in the range of from about 20° C. to about 25° C.

Following pH adjustment, 14-hydroxymorphinone is preferably isolated as a solid.

Preferably, the 14-hydroxymorphinone is isolated according to known methods, for example, by filtration and with optional drying, and/or optional washing of the filtercake with water and a suitably selected organic solvent, for example a suitably selected alcohol, such as isopropanol, n-butanol, ethanol, and the like, preferably isopropanol.

In an embodiment, the isolated 14-hydroxymorphinone is washed with water, in an amount in the range of from about 1 to about 10 kilograms water (relative to one kilogram of CPS oripavine), or any amount or range therein, preferably in an amount in the range of from about 1 to about 5 kilograms water, more preferably in an amount in the range of from about 1 to about 3 kilograms water, more preferably in an amount of about 2 kilograms water.

In another embodiment, the 14-hydroxymorphinone is washed with isopropanol (IPA), in an amount in the range of from about 1 to about 4 liters (relative to one kilogram CPS oripavine), or any amount or range therein, preferably in an amount in the range of from about 1 to about 2 liters, more preferably in an amount of about 1.3 liters.

Preferably, the 14-hydroxymorphinone is washed with a suitably selected organic solvent such an alcohol, preferably isopropanol or dried, for example under elevated temperature and/or vacuum conditions to yield a solid with a water content of less than about 10%, as measured by Karl-Fischer (KF), preferably to a water content of less than about 5%, more preferably to a water content of less than about 3%, more preferably to a water content of less than about 2%, more preferably to a water content less than about 1%.

In an embodiment of the present invention, the oxidation described in step 1 above proceeds according to the following steps:
i. Water is charged to a reaction vessel;
ii. Formic acid is added to the reaction vessel;
iii. CPS oripavine is charged to the vessel to yield a reaction mixture; and stirring initiated to dissolve the CPS oripavine;
iv. EDTA is added to the reaction mixture;
v. the reaction mixture is heated to a temperature in the range of from about room temperature to about 40° C.;
vi. hydrogen peroxide is added to the reaction mixture;
vii. sulfuric acid is added to the reaction mixture;
viii. the reaction mixture is stirred at a temperature less than about 28° C.; wherein the reaction mixture is stirred until the reaction is deemed complete (preferably until HPLC assay sampling indicates consumption of greater than about 95%, preferably greater than or equal to about 98%, more preferably greater than or equal to about 99%, of the original charge of CPS oripavine or conversion of greater than about 95%, preferably greater than or equal to about 98%, more preferably greater than or equal to about 99%, of the original charge of CPS oripavine to 14-hydroxymorphinone);
ix. the reaction is quenched by addition of aqueous sodium sulfite;
x. 14-hydroxymorphinone is precipitated (and optionally isolated) by addition of an organic amine base.

In another embodiment of the present invention, the oxidation described in step 1 above proceeds according to the following steps:
i. Water is charged to a reaction vessel; wherein the water is added in an amount of about 1.5 kg/kg (relative to the mass of CPS oripavine)
ii. formic acid is added to the reaction mixture; wherein the formic acid is added in an amount of about 1.13 kg/kg (relative to the mass of CPS oripavine);
iii. CPS is charged to the vessel and stirring initiated to dissolve the CPS oripavine;
iv. EDTA is added to the reaction mixture; wherein the EDTA is added in an amount of about 0.02 kg/kg (relative to the mass of CPS oripavine);
v. the reaction mixture is heated to a temperature of about 35° C.;
vi. hydrogen peroxide is added to the reaction mixture; wherein the hydrogen peroxide is added in an amount of about 1.14 molar equivalents (relative to the moles of CPS oripavine);
vii. 95% to 98% sulfuric acid is added to the reaction mixture; wherein the sulfuric acid is added in an amount of about 0.17 kg/kg (relative to the mass of CPS oripavine);
viii. the reaction mixture is stirred at a temperature less than about 28° C., until HPLC assay sampling indicates consumption of greater than about 95% (preferably greater than or equal to about 98%, more preferably greater than or equal to about 99%) of the original charge of CPS oripavine or conversion of greater than about 9% (preferably greater than or equal to about 98%, more preferably greater than or equal to about 99%) of the original charge of CPS oripavine to 14-hydroxymorphinone;
ix. aqueous sodium sulfite is added to the reaction mixture; wherein the aqueous sodium sulfite is added in an amount sufficient to quench the reaction (i.e. to neutralize any remaining peroxyacid);
x. 14-hydroxymorphinone is precipitated by addition of an TEA; wherein the TEA is added in an amount sufficient to raise the pH of the reaction mixture to a pH of about 8.5.
xi. 14-hydroxymorphinone is isolated by filtration.

In the second step of a process of the present invention, 14-hydroxymorphinone (preferably prepared as described in Step 1 above) is dissolved in DMF (dimethylformamide) and then reacted with a suitably selected hydrogenating agent, preferably hydrogen gas; in the presence of a suitably selected catalyst such as Pd/C catalyst, and the like; in the presence of a suitably selected proton source such as dibasic potassium phosphate, and the like; and optionally in the presence of a suitably selected metal scavenger such as EDTA, and the like; to yield oxymorphone freebase.

More particularly, 14-hydroxymorphinone is dissolved in DMF; wherein the DMF is present in any amount sufficient to dissolve the 14-hydroxymorphinone; preferably, the DMF is present in an amount in the range of from about 1 L/kg to about 10 L/kg (relative to the amount (mass) of 14-hydroxymorphinone), or any amount or range therein, more preferably in an amount in the range of from about 1 L/kg to about 5 L/kg, more preferably in an amount in the range of from about 2 L/kg to about 6 L/kg, more preferably in an amount in the range of from about 2 L/kg to about 4 L/kg, more preferably in an amount in the range of from about 2.3 L/kg to about 4 L/kg, more preferably in an amount in the range of from about 2.5 L/kg to about 3.5 L/kg, more preferably in an amount of about 3 L/Kg;

and reacted with a suitably selected hydrogenating agent such as hydrogen gas, and the like; wherein the hydrogen gas is preferably present at a pressure in the range of from about 10 psi to about 75 psi, more preferably at a pressure in the range of from about 20 psi to about 50 psi, or any amount or range therein, more preferably at a pressure in the range of from about 30 psi to about 50 psi, more preferably at a pressure in the range of from about 30 psi to about 40 psi, more preferably at a pressure of about 35 psi;

in the presence of a suitably selected catalyst such as Pd/C, and the like, for example 5% Pd/C, 10% Pd/C, and the like, preferably 5% Pd/C; wherein the catalyst is preferably present in an amount in the range of from about 0.5 wt % to about 2 wt % (relative to the amount (weight) of 14-hydroxymorphinone), or any amount or range therein, more preferably present in an amount in the range of from about 1 wt % to about 2 wt %, more preferably in an amount in the range of from about 1.5 wt % to about 2 wt %, more preferably in an amount of about 1.8 wt %;

optionally, in the presence of a suitably selected proton source such as dibasic potassium phosphate, monobasic potassium phosphate, phosphoric acid, and the like, preferably dibasic potassium phosphate or monobasic potassium phosphate, more preferably dibasic potassium phosphate; wherein the proton source is preferably present in an amount in the range of from about 0.001 kg/kg to about 0.05 kg/kg (relative to the amount (weight) of 14-hydroxymorphinone), or any amount or range therein, more preferably in an amount in the range of from about 0.001 kg/kg to about 0.02 kg/kg, more preferably in an amount in the range of from about 0.001 kg/kg to about 0.015 kg/kg, more preferably, in an amount of about 0.01 kg/kg;

and optionally in the present of a suitably selected metal scavenger such as a suitably selected carboxylic acid (such as EDTA, and the like), a sulfonic acid (such as propylsulfonic acid, and the like), an amine (such as tris-(2-aminoethyl)amine, and the like) or a thiol (such as 1-propanethiol, and the like), optionally on a solid support, preferably EDTA; wherein the metal scavenger is present in an amount in the range of from about 0.001 kg/kg to about 0.05 kg/kg (relative to the amount of 14-hydroxymorphinone), or any amount or range therein, more preferably in an amount in the range of from about 0.01 kg/kg to about 0.04 kg/kg, more preferably in an amount in the range of from about 0.01 kg/kg to about 0.03 kg/kg, more preferably in an amount of about 0.02 kg/kg;

preferably at a temperature greater than about room temperature, preferably at a temperature in the range of from about 20° C. to about 50° C., or any temperature or range therein, more preferably at a temperature in the range of from about 25° C. to about 45° C., more preferably at a temperature in the range of from about 30° C. to about 40° C., more preferably at a temperature of about 35° C.; to yield oxymorphone as its corresponding freebase.

In an embodiment of the present invention, the amount of DMF is sufficient to at least partially dissolve the 14-hydroxymorphinone.

One skilled in the art may theorize that the addition of a greater amount of catalyst may result in increased rate of reaction. However, for the process(es) of the present invention, it has been noted that increased catalyst loads also adversely affect product color and/or appearance.

Preferably, the mixture comprising the oxymorphone freebase is filtered to remove the catalyst; and the filtercake rinsed with DMF.

Preferably, the oxymorphone freebase is isolated, for example according to known methods.

In an embodiment, to the reaction mixture comprising oxymorphone freebase (preferably, a mixture of the filtrate resulting from the removal of the catalyst combined with any DMF wash) is added sodium hydrosulfite (to improve color or appearance); wherein the sodium hydrosulfite is added in an amount in the range of from about 0.001 kg/kg to about 0.05 kg/kg (relative to the amount (weight) of 14-hydroxymorphinone), or any amount or range therein, preferably in an amount in the range of from about 0.005 kg/kg to about 0.03 kg/kg, more preferably in an amount in the range of from about 0.008 kg/kg to about 0.02 kg/kg, more preferably in an amount of about 0.01 kg/kg.

The oxymorphone freebase is then preferably precipitated from the reaction mixture by addition of water; wherein the water is added in an amount in the range of from about 6 L/kg to about 12 L/kg (relative to the amount (weight) of 14-hydroxymorphinone), or any amount or range therein, more preferably, in an amount in the range of from about 8 L/kg to about 12 L/kg, more preferably, in an amount in the range of from about 10 L/kg;

and wherein the temperature of the reaction mixture is preferably controlled to a temperature less than about 40° C. (for example, by jacket temperature, rate of addition of water, etc.), more preferably the temperature is controlled to a temperature in the range of from about 10° C. to about 37° C., or any temperature or range therein, more preferably, the temperature is controlled to a temperature in the range of from about 20° C. to about 35° C., more preferably, the temperature is controlled to a temperature in the range of about 30° C. to about 35° C.

It has been observed that addition of the water to the reaction mixture results in an initial exotherm, which raises the temperature of the reaction mixture. Preferably, the temperature of the reaction mixture is allowed to increase with the generated exotherm. More preferably, the temperature of the reaction mixture is allowed to increase to a temperature of less than about 50° C., preferably to a temperature less than about 40° C., preferably to a temperature in the range of from about 30° C. to about 35° C.

The oxymorphone freebase is then preferably isolated, for example, by further cooling the reaction mixture to a temperature less than about 35° C., preferably to a temperature in the range of from about 10° C. to about 30° C., or any temperature or range therein, more preferably to a temperature in the range of from about 15° C. to about 25° C., more preferably to a temperature of about 20° C., and filtered; followed by optional washing of the filtercake with a suitably selected solvent such as water. Preferably, the filtercake (comprising the isolated oxymorphone freebase) is dried, optionally under a temperature greater than room temperature and/or under reduced pressure.

In an embodiment of the present invention, the reduction of 14-hydroxymorphinone described in step 2 above proceeds according to the following steps:
i. 14-hydroxymorphinone is charged to a nitrogen purged hydrogenation vessel;
ii. EDTA is charged to the vessel;
iii. dibasic potassium phosphate is charged to the vessel;
iv. DMF is charged to the vessel;
v. the catalyst is charged to the vessel;
vi. H₂ gas is introduced into the reaction vessel at 35 psi and 35° C. to effect hydrogenation of the 14-hydroxymorphinone; wherein the hydrogenation is continued until the reaction is deemed complete (preferably until HPLC assay sampling indicates consumption of greater than about 95%, preferably greater than or equal to about 98%, more preferably greater than or equal to about 99% of the original charge or conversion of greater than about 95%, preferably greater than or equal to about 98%, more preferably greater than or equal to about 99% of the original charge of 14-hydroxymorphinone to oxymorphone freebase); to yield oxymorphone freebase;
vii. the reaction mixture comprising the oxymorphone freebase is filtered to remove the catalyst;
viii. sodium hydrosulfite is added to the filtrate;
ix. water is added to the filtrate to yield oxymorphone freebase is a precipitate;
x. the oxymorphone freebase precipitate optionally isolated by filtration and optionally dried.

In another embodiment of the present invention, the reduction of 14-hydroxymorphinone described in step 2 above proceeds according to the following steps:
i. 14-hydroxymorphinone is charged to a nitrogen purged hydrogenation vessel;
ii. EDTA is charged to the vessel; wherein the EDTA is added in an amount of about 0.02 kg/kg (relative to the mass of 14-hydroxymorphinone);
iii. dibasic potassium phosphate is charged to the vessel; wherein the dibasic potassium phosphate is added in an amount of about 0.01 kg/kg (relative to the mass of 14-hydroxymorphinone);
iv. DMF is charged to the vessel; wherein the DMF is added in an amount of about 3 L/kg (relative to the mass of 14-hydroxymorphinone);
v. 5% Pd/C catalyst is charged to the vessel; wherein the catalyst is added in an amount of about 0.018 kg/kg (relative to the mass of 14-hydroxymorphinone);
vi. H₂ gas is introduced into the reaction vessel at 35 psi and 35° C. to effect hydrogenation of the 14-hydroxymorphinone; wherein the hydrogenation is continued until the HPLC assay sampling indicates consumption of greater than about 95% (preferably greater than or equal to about 98%, more preferably greater than or equal to about 99%) of the original charge of 14-hydroxymorphinone or conversion of greater than about 95% (preferably greater than or equal to about 98%, more preferably greater than or equal to about 99% of the original charge of 14-hydroxymorphinone to oxymorphone freebase; to yield oxymorphone freebase;
vii. the reaction mixture is filtered to remove the catalyst;
viii. sodium hydrosulfite is added to the filtrate; wherein the sodium hydrosulfite is added in an amount of about 0.01 kg/kg (relative to the mass of 14-hydroxymorphinone);
ix. water is added to the filtrate; wherein the water is added in an amount of about 10 L/kg (relative to the mass of 14-hydroxymorphinone); to yield oxymorphone freebase as a precipitate;
x. the oxymorphone freebase precipitate is optionally isolated by filtration and optionally dried.

In an embodiment, the present invention is directed to a process for the hydrogenation of 14-hydroxymorphinone to oxymorphone freebase, wherein the yield of the oxymorphone freebase (product) is greater than about 80%, preferably greater than about 85%, more preferably greater than about 88%, for example, in the range of from about 80% to about 100%, more preferably, in the range of from about 88% to about 92%, more preferably, about 90%.

### Conversion to HCl Salt

In an embodiment of the present invention, the oxymorphone freebase is reacted with a suitably selected acid, preferably HCl, to yield the corresponding acid addition salt, preferably the corresponding pharmaceutically acceptable salt.

In an embodiment, the oxymorphone freebase is dissolved in a mixture of water and a suitably selected alcohol, such as ethanol, methanol, and the like, preferably ethanol; with heating to a temperature of about 50° C.; to the reaction mixture is then added hydrochloric acid (HCl); wherein the amount of HCl added is sufficient to adjust the pH of the resulting mixture to about pH 1; and the resulting mixture heated to a temperature of about 50° C.; to yield oxymorphone hydrochloride.

The reaction mixture comprising oxymorphone hydrochloride is optionally charged with powdered activated carbon and filtered.

To the reaction mixture comprising oxymorphone hydrochloride (or the filtrate following treatment with activated carbon and filtration) is then added isopropanol; wherein the isopropanol has been heated to about 50° C. The resulting mixture is then cooled, preferably to a temperature of less than about room temperature, more preferably to about 20° C.; to yield a precipitate of oxymorphone hydrochloride. Optionally, the reaction mixture comprising oxymorphone hydrochloride is seeded with the desired crystalline form of oxymorphone hydrochloride.

The oxymorphone hydrochloride is preferably isolated and dried according to known methods, for example, by cooling filtration, with optional washing of the filtercake with a suitable selected, cooled solvent, for example isopropanol at 20° C. The isolated oxymorphone hydrochloride is preferably dried (optionally under vacuum) in the presence of water.

### Advantage of the Process(es) of the Present Invention (Organic Impurities):

One skilled in the art will recognize that in Step 1 (wherein CPS oripavine is oxidized to 14-hydroxymorphinone), residual peroxide can lead to increased levels of organic impurities such as 8-hydroxyoxymorphone and/or 2,2-bis-oxymorphone. Additionally, any residual (unreacted) CPS oripavine would be expected to convert to the undesired organic impurity hydromorphone.

In the process(es) of the present invention, 14-hydroxymorphinone is isolated as a solid (by addition of an organic amine base) and any residual peroxide is either decomposed (by reacting with for example, aqueous sodium sulfite) or removed during isolation, thereby limiting (preferably minimizing) the formation of 8-hydroxyoxymorphone and/or 2,2′-bis-oxymorphone. Further, isolation of 14-hydroxymorphinone reduces the amount of residual (unreacted) CPS oripavine which is present in the reaction mixture, thereby limiting the amount of hydromorphone (an organic impurity) which is produced.

One skilled in the art will further recognize that in Step 2 (wherein 14-hydroxymorphinone is hydrogenated to oxymorphone free base), acidic solvent can lead to the formation of the undesired organic impurity 14-hydroxydihydromorphine (α). In the process(es) of the present invention, 14-hydroxymorphinone is dissolved in for example, dimethylformamide, without the addition of acid thereby limiting (preferably minimizing) the formation of 14-hydroxydihydromorphine (α).

Thus, at least one advantage of the process(es) of the present invention is a decrease in the amount of organic impurities present in the final isolated oxymorphone freebase product.

### Advantage of the Process(es) of the Present Invention (Inorganic Impurities):

One skilled in the art will recognize that in one known process for the conversion of CPS oripavine to oxymorphone, CPS oripavine is dissolved in acidic water and the oxymorphone is precipitated using sodium hydroxide, resulting in the formation of inorganic salts as byproducts.

In the process(es) of the present invention, 14-hydroxymorphinone is isolated as a solid using an organic amine base, for example triethylamine, and the hydrogenation of 14-hydroxymorphinone is carried out in DMF and the oxymorphone freebase is precipitated from water, without the need for additional acid or base, resulting in a decrease in the formation of inorganic salts (impurities) remaining in the final oxymorphone freebase product.

### SYNTHESIS EXAMPLES

The following Examples are set forth to aid in the understanding of the invention, and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

Examples 1-4 describe recipes/procedures for the synthesis of the title compounds. One or more batches of the said compounds were prepared according to the recipes/procedures as described below. The physical properties (e.g. MS⁺, ¹H NMR, etc.) listed at the end of the synthesis descriptions below are the physical properties measured for a representative sample of the prepared compound, from a batch prepared according to the described procedure and/or are a range of measured values from multiple batches run according to the procedure as described.

### Example 1

### Recipe/Procedure for Synthesis of Oxymorphone Freebase

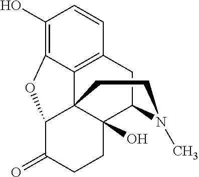

### Step 1: Synthesis of 14-Hydroxymorphinone (14-HM)

CPS oripavine (contained wt.), water (1.0 kg/kg relative to oripavine), and formic acid (98%, 1.04 kg/kg relative to oripavine) were charged to a reactor. The resulting suspension was agitated, heated to a temperature in the range of 30-40° C., and held for 30-60 minutes. The resulting mixture was filtered while maintaining temperature in the range of 30-40° C., to remove any insoluble matter. The filtercake was washed with water (0.6 L/kg relative to oripavine). The water wash was added to the filtrate and the combined mixture added to a reactor. EDTA (ethylenediaminetetraacetic acid, 0.02 molar equivalents relative to oripavine) was charged into the reactor and the solution was cooled to 20-25° C. Hydrogen peroxide (30%, 1.08 molar equivalents relative to starting CPS oripavine) was dosed to the reaction solution over 30-60 minutes. Sulfuric acid (98%, 0.17 kg/kg relative to oripavine) was charged to the reactor while maintaining temperature at 20-25° C. The reaction mixture was then stirred for approximately 14-16 hours, with total time determined by an in-process test to attain a level of not less than (NLT) 99.5% w/w of 14-hydroxymorphinone to oripavine. Once the reaction was deemed “complete”, the reaction was quenched with aqueous sodium sulfite solution (0.08 molar equivalents in 0.28 L/kg of water relative to oripavine) while maintaining the temperature at 15-25° C. The reaction mixture was agitated for a minimum of 30 minutes and then diluted with water (5.0 kg/kg relative to oripavine). Triethylamine was added to attain a pH of 8.6-9.2, while maintaining the temperature at 20-32° C., to precipitate the product. The resulting slurry was cooled to 20-25° C. and held for a minimum of 60 minutes. The product (14-Hydroxymorphinone, 14-HM) was filtered and washed with water (1.0 kg/kg relative to oripavine), then washed with isopropanol (1.3 L/kg relative to oripavine) to yield 14-hydroxymorphinone as a solid. Optionally, the wet product was dried at a temperature in the range of 35-45° C., under vacuum with a slight air sweep. Typical yield (based on multiple runs of this reaction step) was in the range of 85-90%.

### Step 2: Synthesis of Oxymorphone Freebase OMFB

14-Hydroxymorphinone (14-HM), dimethylformamide (DMF, 3.0 L/kg relative to 14-HM), ethylenediaminetetraacetic acid (EDTA, 0.02 kg/kg relative to 14-HM), potassium phosphate dibasic (0.01 kg/kg relative to 14-HM), and Pd/C (5%, 0.011 kg/kg relative to 14-HM) were charged to a hydrogenator. The reaction mixture was hydrogenated at a temperature in the range of 25-45° C. and at a hydrogen pressure in the range of 30-50 psi until the reaction was deemed “complete”. Once the reaction was complete, the catalyst was removed by filtration and washed with dimethylformamide (DMF, 1.0 L/kg relative to 14-HM). Sodium hydrosulfite (0.01 kg/kg relative to 14-HM) was charged and the reaction mixture cooled to a temperature in the range of 10-35° C. The oxymorphone base was precipitated by adding water (10 L/kg relative to 14-HM) while maintaining a temperature below 40° C. The reaction mixture was then cooled to a temperature in the range of 15-25° C. and agitated for 30-60 minutes. Oxymorphone base (OMFB) was filtered and washed with water (3.0 L/kg relative to 14-HM). The wet product was dried at a temperature in the range of 50-60° C., under vacuum with slight air sweep. Typical yield (based on multiple runs of this reaction step) was in the range of 85-90%.

### Example 2

### Synthesis of Oxymorphone Freebase

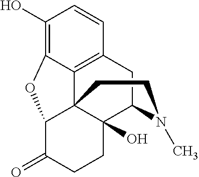

### Step 1: Synthesis of 14-Hydroxymorphinone (14-HM)

CPS oripavine (200 g, contained), water (200 mL), and formic acid (98%, 170 mL) were charged to a reactor. The suspension was agitated, heated to 37.3° C., and held for 52 minutes. The reaction was filtered to remove any insoluble matter. The filtercake was washed with water (120 mL). The water wash was added to the filtrate and the combined mixture added to a reactor. Ethylenediaminetetraacetic acid (EDTA, 3.94 g) was charged into the reactor and the solution was cooled to 21.5° C. Hydrogen peroxide (30%, 82.44 g) was dosed to the reaction solution over 62 minutes. Sulfuric acid (98%, 33.98 g) was charged to the reactor while maintaining 21.9-24.3° C. The reaction mixture was stirred for 15 hours with an in-process test to attain a level of NLT 99.5% w/w of 14-hydroxymorphinone to oripavine. Once the reaction was complete, the mixture was quenched with aqueous sodium sulfite solution (6.78 g in 56 mL water) while maintaining 19.2-21.1° C. The reaction was agitated for 30 minutes and then diluted with water (945 mL). Triethylamine was added to pH 8.76 while maintaining 22.0-24.6° C. to precipitate the product. The reaction slurry was held for 77 minutes. The product (14-HM) was filtered and washed with water (200 mL), then washed with isopropanol (260 mL). The wet product was dried at ˜50° C. under vacuum with a slight air sweep. The 14-HM yield was 85%.

### Step 2: Synthesis of Oxymorphone Base

14-Hydroxymorphinone (14-HM, 150.0 g), dimethylformamide (DMF, 450 mL), ethylenediaminetetraacetic acid (EDTA, 3.01 g), potassium phosphate dibasic (1.49 g), and Pd/C (5%, 1.65 g) were charged to the hydrogenator. The reaction was hydrogenated at 35° C. and 35 psi until the reaction was complete. Once the reaction was complete, the catalyst was removed by filtration and washed with dimethylformamide (DMF, 170 mL). Sodium hydrosulfite (1.50 g) was charged and the reaction was cooled to 15° C. The product was precipitated by adding water (1500 mL) while maintaining a temperature below 31° C. The reaction was cooled to 10-20° C. and agitated for 49 minutes. The product (OMFB) was filtered and washed with water (490 mL). The wet product was dried at 50° C. under vacuum with slight air sweep. The oxymorphone base yield was 90%. Table 1 below, lists physical properties for the starting material, 14-hydroxymorphinone intermediate and isolated oxymorphone base for the material prepared as described in Example 2.

TABLE 1
CPS Oripavine, 14-Hydroxymorphinone (14-HM) and
Oxymorphone Freebase Physical Properties

| Test Results | CPS | 14-HM | OMFB |
|---|---|---|---|
| Loss On Drying (%) | | 0.3 | 1.2 |
| Residue On Ignition (%) | 15 | 0.00 | 0.01 |
| Residual Organic Solvent (DMF) | | | 761 |
| Residual Organic Solvent (TEA) | | | ND |
| Residual Organic Solvent (IPA) | | | ND |
| KF (%) | | 0.18 | 0.95 |
| Assay (% w/w) (Wet Basis) | | 100.1 | 98.6 |
| Assay (% w/w) (Dry Basis) | 86 | | 99.7 |
| Area (%) | 101.6 | 98.58 | 100 |
| Color | Tan | Yellow | White |

HPLC Impurities (% area)

| | | |
|---|---|---|
| Unknown 1 at RRT 0.26 | 0.2 | |
| Unknown 7 at RRT 0.81 | 0.3 | |
| Unknown 8 at RRT 0.92 | 0.1 | |
| Unknown 9 at RRT 1.78 | 0.1 | |
| Unknown 10 at RRT 1.86 | 0.1 | |
| Oxymorphone-N-Oxide | | <0.05 |
| 8-Hydroxyoxymorphone | | <0.05 |
| Hydromorphone | | 0.05 |
| 14-Hydroxymorphinone | | <0.05 |
| 14-Hydroxydihydromorphine (α) | | <0.05 |
| Oxycodone | | 0.11 |
| 2,2′-Bisoxymorphone | | <0.05 |
| Individual Unspecified (Single | | <0.05 |
| Largest) | | |
| Total Impurities | | 0.2 |

### Example 3

### Recipe/Procedure for Synthesis of Oxymorphone Freebase

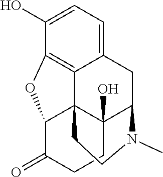 [SMILES: CN1CC[C@]23c4c5ccc(O)c4O[C@H]2C(=O)CC[C@@]3(O)[C@H]1C5]

### Step 1: Synthesis of 14-Hydroxymorphinone (14-HM)

A reactor vessel was purged with nitrogen. CPS oripavine (250-350 kg), water (1.4-1.8 L/kg relative to oripavine), formic acid (90%, 1.1-1.22 kg/kg relative to oripavine) and ethylenediaminetetraacetic acid (EDTA, 0.02 kg/kg relative to oripavine) were charged to a reactor. The resulting suspension was agitated, heated to a temperature in the range of 30-40° C., and held for 30-60 minutes. The temperature of the reaction mixture was then adjusted to a temperature in the range of 20-28° C. Hydrogen peroxide (1-1.2 molar equivalents relative to starting CPS oripavine) at a temperature in the range of 20-28° C. was dosed to the reaction solution over 30-120 minutes, and the reaction mixture agitated. Sulfuric acid (98%, 0.13-0.2 kg/kg relative to oripavine) was charged to the reaction mixture while maintaining the temperature at 20-28° C. The reaction mixture was then stirred for approximately 10-12 hours, with total time determined by an in-process test to attain a level of not less than (NLT) 99.5% w/w of 14-hydroxymorphinone to oripavine. Once the reaction was deemed “complete”, the reaction was quenched with aqueous sodium sulfite solution (0.02-0.08 molar equivalents relative to oripavine in 0.53 L/kg water) over 20-40 minutes, while maintaining the temperature at 18-22° C. The reaction mixture was agitated for 30-40 minutes and then diluted with water (3-6 L/kg relative to oripavine). Water (3.0-6.0 L/kg relative to oripavine) was added. The pH of the reaction mixture was adjusted to a pH in the range of 8.2-8.7 (by addition of triethylamine), and the reaction mixture held with stirring at this pH for about 15 minutes. The pH was further adjusted to a final pH in the range of pH 8.6-9.2 (by addition of triethylamine), and the reaction mixture held, with agitation of an additional 1-23 hours to yield 14-hydroxymorphinone as a precipitate. The precipitate was isolated by centrifuge, the wet cake washed with water (81-85 kg) and isopropanol (81-85 kg) and dried at 35-45° C. under 25″ hg vacuum.

### Step 2: Synthesis of Oxymorphone Freebase OMFB

14-Hydroxymorphinone (14-HM 200-315 kg), ethylenediaminetetraacetic acid (EDTA, 0.02 kg/kg relative to 14-HM) and potassium phosphate dibasic (0.01 kg/kg relative to 14-HM) were charged to a hydrogenator. Nitrogen gas was added to inert the reactor. Dimethylformamide (DMF, 2.3-4 L/kg relative to 14-HM) was then added and the reaction mixture agitated for 25-30 min. Pd/C (5%, 0.01-0.02 kg/kg relative to 14-HM) catalyst was charged to a hydrogenator. The reaction mixture was hydrogenated at a temperature in the range of 25-45° C. and at a hydrogen pressure in the range of 30-40 psi until the reaction was deemed “complete” (about 2 hours), with total time determined by an in-process test to attain a level of not less than (NLT) 99.5% w/w of oxymorphone freebase to 14-hydroxymorphinone. Once the reaction was complete, the catalyst was removed by filtration and washed with dimethylformamide (DMF, 1-2 L/kg relative to 14-HM). Nitrogen gas was added to inert the reactor. Sodium hydrosulfite (0.01 kg/kg relative to 14-HM) was charged, nitrogen gas added to inert the reactor, followed by addition of water (9.5-10.5 L/kg (relative to 14-HM) at 20-30° C. The reaction mixture cooled to a temperature in the range of 15-25° C. and held at this temperature for 1-15 hours. Addition of water resulted in the precipitation of the oxymorphone freebase, which was isolated by centrifuge and dried at 40-60° C. under 25″ hg vacuum.

### Example 4

### Preparation of 14-Hydroxymorphinone from Thebaine

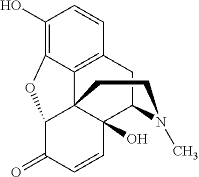

### Step 1: Preparation of 14-Hydroxycodeinone

To thebaine (100 g) in a 1 L reactor under nitrogen atmosphere at 20-25° C. was added water (145 g). To the resulting suspension was added formic acid (98-100%, 100 5) under stirring. The addition was observed to be exothermic. The resulting dark brown mixture was stirred at 20-25° C. for 30-60 min until a clear solution was obtained. To the resulting brown clear solution was added a solution of 35% hydrogen peroxide (34.7 g, 357 mmol, 1.15 mol-eq) in water (15 g) slowly, over 30 min, at 20-25° C. and with stirring. The resulting mixture was stirred at 23-28° C. for 21 hours. The reaction was deemed complete as determined by HPLC where the area % of starting material (thebaine) was not more than 0.25 area %. To the mixture was then added sodium sulfite (5.89 g, 46.7 mmol, 1.15 mol-eq) freshly dissolved in water (51.0 g) at 20-30° C. over 30 min, and the reaction mixture stirred for 30 min at 20-25° C. (in order to decompose any remaining H₂O₂ in the mixture). Water was added (52.5 g). Triethylamine (waterfree, 219 g, 2.16 mol, 0.99 mol-eq to formic acid) was then added (addition is exothermic) as follows. The ⅓ portion was added over 15-20 min until the internal temperature of the reaction mixture reached 45° C. The second ⅓ portion was added slowly, at a rate which maintained the internal temperature at 45-50° C. (about 1.5 hours) to a pH of 6-6.5. The resulting light yellow suspension was then stirred at 45° C., pH 6.3 for 1.5 hours, then cooled to 23° C. The final 1.3 portion was then added over 30-60 min to a pH of 9.5. The resulting mixture was stirred at 23° C. for 30 min and the pH checked to make certain it was in the range of pH 9-9.5. The mixture was filtered, the wet cake washed with water (182 g) and twice with methanol (180 g). The resulting wet, almost white solid was dried at 90° C. in vacuo over 20 hours (until the water content was less than 0.5%) to yield 14-hydroxycodeinone.

Yield: 92.4 g, 93.8% assay corrected

HPLC purity: 99.5 Area %

HPLC Assay: 99.2%

Water content: 0.1% KF

### Step 2: Preparation of 14-Hydroxymorphinone

Nitrobenzene (water content 0.34% KF, 275 g) was added into a 350 mL reaction. The jacket temperature was increased to 115° C. and 25 mg were distilled. Water (0.327 g) was added to set the water content to 0.27 wt %. Aluminum chloride (powder, 67.1 g) was added in portions resulting in an exothermic reaction. The reaction mixture was then heated to 50-53° C. until a clear solution was obtained (about 30 min) and the mixture was then cooled to 25° C. 14-Hydroxycodeeinone (50.0 g) was added in 10 portions during 30 minutes at 22-26° C. The resulting dark brown mixture was heated to 30° C. over 10 min, then stirred at 30° C. for 60 min. The mixture was then slowly heated to 70° C. over 60 min and stirred at 70° C. for 14 hrs. Once the reaction was deemed complete as determined by HPLC (not more than 0.50 area % of starting material, 14-hydroxycodeinone) the reaction mixture was cooled to 25° C. over 30 min. To the mixture was then added water (332 g) and acetone (33.2 g) at a jacket temperature of −10° C. (internal temperature of 0-5° C.). The dropping funnel was washed with nitrobenzene (12.5 g) and added to the reaction. Once the mixture is quenched, the temperature of the brown greenish suspension was increased to 50-55° C. over 60 min, then decreased to 20-25° C. and stirred for 60 min. The mixture was then filtered and the filter cake pressed and washed with methyl-isobutyl ketone (62.5 g) to yield a wet solid. The wet solid was charged back to the reaction and methanol (75 g) was added. The resulting suspension was heated to 60-65° C. for 1 hour. The resulting suspension was then slowly cooled to −5° C. over about 2 hours, then stirred at −5° C. for 60 min, then filtered and the filter cake washed with acetone (2×20 g, precooled to −5° C.). The wet cake was charged into the reactor, suspended in water (376 g) and acetic acid (14 g). The pH of the reaction mixture was adjusted to a pH in the range of 4-8-5.0 with triethylamine. The mixture was stirred until an almost clear, slightly turbid solution was formed. To the dark green mixture was then added activated carbon NORIT CN1 (2.5 g) and the mixture stirred at 25° C. for 1-2 hours and filtered. The filter residue was washed with water (2×25 g). Additional triethylamine was slowly added to the filtrate to a pH in the range of 8.8-92-0.2 at a temperature of 20-25° C., over about 30 min. The resulting suspension was stirred at 20-25° C. for 660 min, the pH controlled at about pH 9.15 (adjusting with triethylamine of conc. HCl, as necessary). The resulting mixture was filtered and the filter cake washed with water (75 g). The wet cake was dried at 80-85° C. in vacuo over the weekend to yield 14-hydroxymorphinone.

Yield: 33.8 g, 71.7% assay corrected

HPLC purity: 99.6 Area %

HPLC Assay: 99.8%

Water content: 0.2% KF

### Example 5

### Prophetic Example

### Preparation of 14-Hydroxymorphone and Filtration of CELITE

In the first step of the process of the present invention, concentrated poppy straw (CPS) oripavine (also known as 6,7,8,14-tetradehydro-4,5α-epoxy-6-methoxy-17-methylmorphinan-3-ol) is oxidized to yield 14-hydroxymorphinone (14-HM). The modified procedure captured below provides an option to filter CELITE (which is introduced into the reaction with the CPS) after the oxidation reaction is complete and before isolation of 14-hydroxymorphinone.

CPS Oripavine (in a mixture with CELITE), water and 90% w/w or 98% w/w formic acid are charged to a reactor vessel. The suspension is agitated and heated to 30-40° C. The slurry is held at this temperature for 30-60 minutes and then cooled to 20-25° C.

Ethylenediaminetetraacetic acid (EDTA, 0.02 molar equivalents relative to Oripavine) is charged into the reactor. A charge of hydrogen peroxide is dosed to the Oripavine solution over 30-60 minutes, while maintaining a reaction temperature of 20-25° C. Sulfuric acid (95-98%) is added after the addition of hydrogen peroxide. Sulfuric acid is charged into the reactor at a rate that maintains a temperature below 25° C. The reaction mixture is stirred for approximately 12-14 hours with an in-process test to attain a level of NLT (not less than) 99.5% w/w (equal to approximately 98 Area %) of product.

Once the reaction is complete, the mixture is quenched with aqueous sodium sulfite solution at a rate that maintains a temperature of 20-25° C., over 20-30 minutes. The reaction slurry is agitated for approximately 30 minutes and diluted with water.

The reaction mixture is heated to 40-45° C. to dissolve the product, leaving the insoluble CELITE to be filtered as hot filtration to ensure product solubility in the reaction mixture. After CELITE filtration, the reaction mixture is cooled to room temperature.

The product (14-Hydroxymorphone) is precipitated with the addition of triethylamine to the desired pH in the range of 8.6 to 9.2. The batch temperature is maintained below 30° C. through both jacket temperature control and controlled addition of the triethylamine. The reaction slurry is held at room temperature for 30 to 60 minutes, and is then filtered. The cake is washed with water and isopropanol. Optionally, the wet product is dried at 50° C. under vacuum with a slight air sweep. The product (14-HM) is taken forward into further reaction steps, as desired.

### Example 6

### Prophetic Example

### Preparation of 14-Hydroxymorphone and Filtration of CELITE

In the first step of the process of the present invention, concentrated poppy straw (CPS) oripavine (also known as 6,7,8,14-tetradehydro-4,5α-epoxy-6-methoxy-17-methylmorphinan-3-ol) is oxidized to yield 14-hydroxymorphinone (14-HM). The modified procedure captured below provides an option to filter CELITE (which is introduced into the reaction with the CPS) after the oxidation reaction is complete and before isolation of 14-hydroxymorphinone.

CPS Oripavine (in a mixture with CELITE), water and 90% w/w or 98% w/w formic acid are charged to a reactor vessel. The suspension is agitated and heated to 30-40° C. The slurry is held at this temperature for 30-60 minutes and then cooled to 20-25° C.

Ethylenediaminetetraacetic acid (EDTA, 0.02 molar equivalents relative to Oripavine) is charged into the reactor. A charge of hydrogen peroxide is dosed to the Oripavine solution over 30-60 minutes, while maintaining a reaction temperature of 20-2° C. Sulfuric acid (95-98%) is added after the addition of hydrogen peroxide. Sulfuric acid is charged into the reactor at a rate that maintains a temperature below 25° C. The reaction mixture is stirred for approximately 12-14 hours with an in-process test to attain a level of NLT (not less than) 99.5% w/w (equal to approximately 98 Area %) of product.

Once the reaction is complete, the mixture is quenched with aqueous sodium sulfite solution at a rate that maintains a temperature of 20-25° C., over 20-30 minutes. The reaction slurry is agitated for approximately 30 minutes. The reaction mixture is heated to 45-50° C. to dissolve the product, leaving the insoluble CELITE to be filtered as hot filtration to ensure product solubility in the reaction mixture. After CELITE filtration, the reaction mixture is cooled to room temperature and diluted with water and product precipitation is followed.

The product (14-HM) is precipitated with the addition of triethylamine to the desired pH in the range of 8.6 to 9.2. The batch temperature is maintained below 30° C. through both jacket temperature control and controlled addition of the triethylamine. The reaction slurry is held at room temperature for 30 to 60 minutes, and is then filtered. The cake is washed with water and isopropanol. Optionally, the wet product is dried at 50° C. under vacuum with a slight air sweep. The product (14-Hydroxymorphone) is taken forward into further reaction steps, as desired.

The HPLC and UPLC methods of the present invention may be run on any suitably HPLC or UPLC system. Suitably examples include but are not limited to UPLC Waters H-Call, Agilent 1290, Agilent 1100, Agilent 1200, Agilent 1260, Water Alliance 2695, and the like.

In the HPLC and UPLC methods of the present invention, at least one mobile phase is a buffer such as a mixed phosphate buffer in deionized water, an ammonium formate buffer in water or equivalent; wherein the pH of the buffer is controlled to effect separation of the product and impurity peaks. Wherein the buffer is a mixed phophate buffer in water, the pH of the buffer is adjusted and/or controlled by the ratio of the mono-potassium phophate and di-potassium phosphate salts dissolved in the water.

### Example 7

### UPLC Method for Product and Impurity Analysis

### For Analysis of, for Example, Oxymorphone and Salts Thereof

Product impurity profiles were determined using a Water Acquity UPLC system and the following parameters: (a) Column: Waters Acquity UPLC or equivalent equipped with a BEH C18, 100×2.1 mm, 1.7 μm (PART #186002352) column or equivalent, preferably with a Waters Column Stabilized (PART #205000489); (b) Injection Volume: 0.8 μm, (c) Column Temperature: 75° C.; (d) Sample Loop Size: 2 μL; (e) Loop Option: Partial Loop with Needle Overfill; (f) Wavelength Mode: Single; (g) Detector Wavelength: 280 mm; (h) Degasssing: Continuous; (i) Sampling Rate: 10 pt./sec; (j) Filter Time Constant: Normal—0.2 sec; (k) Flow Rate: 0.60 mL/min; (I) Run Time: 10 Min; and using the Gradient Timetable in Table 2, below.

TABLE 2
Gradient Timetable

Mobile
Phase

| Time (min.) | Flow Rate (mL/min.) | % A | % B |
|---|---|---|---|
| Initial | 0.60 | 95 | 5 |
| 1.00 | 0.60 | 84 | 16 |
| 2.00 | 0.60 | 84 | 16 |
| 4.00 | 0.60 | 70 | 30 |
| 6.00 | 0.60 | 65 | 35 |
| 8.50 | 0.60 | 45 | 55 |
| 10.00 | 0.60 | 95 | 5 |

Mobile Phase A was 10 mM mixed phosphate buffer in deionized water prepared by mixing 0.050±0.005 g potassium dihydrogen phosphate (KH₂PO₄) and 1.678±0.025 g dipotassium hydrogen phosphate (K₂HPO₄) in 1000 mL of HPLC Grade water and sonicating the resulting mixture to ensure that the buffer salts are dissolved. After dissolution, pH of the mobile phase is measured to confirm that it is in the range of pH 8.3-8.4.

Mobile Phase B was HPLC Grade (or purer) acetonitrile.

Retention times for selected products and impurities using the method described above, and Gradient Table 2 were as listed in Table 3, below.

TABLE 3
Retention Times using Gradient Table 2

| | | Retention |
|---|---|---|
| | Retention | Time (min) |
| | Time | Relative to |
| Analyte | (min) | Oxymorphone |
| Oxymorphone-N-Oxide | 0.7-0.8 | 0.21 |
| 10-Ketoomorphone HCl | 1.0-1.1 | 0.29 |
| 10-Hydroxyoxymorphone HCl | 1.5-1.6 | 0.43 |
| 8-Hydroxyoxymorphone HCL | 1.8-1.9 | 0.51 |
| Hydromorphone HCl | 2.4-2.5 | 0.68 |
| 14-Hydroxydihydromorphine HCl (α} | 3.1 | 0.88 |
| Oxymorphone HCl | 3.5-3.7 | 1.00 |
| Oxycodone Hydrochloride | 5.4-5.5 | 1.51 |
| 2,2′-Bisoxymorphone Hydrochloride | 5.7-5.9 | 1.61 |
| Pyran Bridged Oxymorphone Dimer HCl | 6.7-6.8 | 1.88 |
| Pyran Bridged Oxymorphone Dimer 2HCl | 7.4-7.5 | 2.07 |

### Example 8

### HPLC Method for Product and Impurity Analysis

### For Analysis of, for Example, Oxycodone and Salts Thereof

Product impurity profiles may be determined using a Water Acquity UPLC system and the following parameters: (a) Column: Waters XBridge HPLC C18, 150×3.0 mm, 3.5 μm (PART #186003028) column or equivalent; (b) Injection Volume: 7.5 μL; (c) Column Temperature: 68° C.; (d) Detector Wavelength: 284 mm; (e) Flow Rate: 0.6 mL/min; (f) Run Time: 30 Min; and using the Gradient Timetable in Table 4, below.

TABLE 4
Gradient Timetable

Mobile
Phase

| Time (min.) | % A | % B | Gradient Change |
|---|---|---|---|
| 0.00 | 96 | 4 | N/A |
| 1.00 | 86 | 14 | Linear |
| 3.00 | 76 | 24 | Linear |
| 8.00 | 70 | 30 | Linear |
| 12.00 | 70 | 30 | Linear |
| 18.00 | 60 | 40 | Linear |
| 21.00 | 40 | 60 | Linear |
| 26.00 | 40 | 60 | Linear |
| 26.10 | 96 | 4 | Step @26 minutes |
| 30.00 | 96 | 4 | Linear |

Mobile Phase A was 10 mM mixed phosphate buffer in deionized water prepared by mixing 0.050±0.005 g potassium dihydrogen phosphate (KH₂PO₄) and 1.678±0.025 g dipotassium hydrogen phosphate (K₂HPO₄) in 1000 mL of HPLC Grade water and sonicating the resulting mixture to ensure that the buffer salts are dissolved. After dissolution, pH of the mobile phase is measured to confirm that it is in the range of pH 8.3-8.4.

Mobile Phase B was HPLC Grade (or purer) acetonitrile.

Retention times for representative product(s) and impurities using the method described above, and Gradient Table 4 were as listed in Table 5, below.

TABLE 5
Retention Times using Gradient Table 4

| | Retention | Retention Time (min) |
|---|---|---|
| Analyte | Time (min) | Relative to Oxycodone HCl |
| Oxycodone-N-Oxide | 5.0-5.2 | 0.41 |
| 14-Hydroxycodeinone HCl | 12.3-12.7 | 0.89 |
| Oxycodone HCl | 10.9-11.0 | 1.00 |
| 7-Methyloxycodone HCl | 17.1-17.3 | 1.38 |

The method as described in Example 8 is also useful for the analysis of benzhydrocodone.

### Example 9

### UHPLC Method for Product and Impurity Analysis

### For Analysis of, for Example, Oxycodone and Salts Thereof

Product impurity profiles may be determined using a Water Acquity UPLC system and the following parameters: (a) Column: Waters Acquity UPLC BEH C18, 100×2.1 mm, 1.7 μm (PART #186002352) column or equivalent, preferably with a Waters Column Stabilized (PART #205000489); (b) Injection Volume: 1.6 μL; (c) Column Temperature: 68° C.; (d) Detector Wavelength: 284 mm; (e) Flow Rate: 0.60 mL/min; (I) Run Time: 9 Min; and using the Gradient Timetable in Table 6, below.

TABLE 6
Gradient Timetable

Mobile
Phase

| Time (min.) | % A | % B | Gradient Change |
|---|---|---|---|
| Initial | 96 | 4 | N/A |
| 0.30 | 86 | 14 | Linear |
| 0.85 | 76 | 24 | Linear |
| 2.40 | 70 | 30 | Linear |
| 3.70 | 70 | 30 | Linear |
| 5.00 | 60 | 40 | Linear |
| 6.50 | 40 | 60 | Linear |
| 7.50 | 40 | 60 | Linear |
| 9.00 | 96 | 4 | Ste @ 7.5 min |

Mobile Phase A was 10 mM mixed phosphate buffer in deionized water prepared by mixing 0.050±0.005 g potassium dihydrogen phosphate (KH₂PO₄) and 1.678±0.025 g dipotassium hydrogen phosphate (K₂HPO₄) in 1000 mL of HPLC Grade water and sonicating the resulting mixture to ensure that the buffer salts are dissolved. After dissolution, pH of the mobile phase is measured to confirm that it is in the range of pH 8.3-8.4.

Mobile Phase B was HPLC Grade (or purer) acetonitrile.

Retention times for selected products and impurities using the method described above, and Gradient Table 6, were as listed in Table 7, below.

TABLE 7
Retention Times using Gradient Table 6

| | Retention | Retention Time (min) |
|---|---|---|
| Analyte | Time (min) | Relative to Oxycodone HCl |
| Oxymorphone-N-Oxide | 1.0-1.1 | 0.31 |
| 14-Hydroxycodeinone HCl | 3.0-3.1 | 0.88 |
| Oxycodone HCl | 3.3-3.5 | 1.00 |
| 7-Methyloxycodone HCL | 4.7-4.8 | 1.40 |

### Example 10

### UHPLC Method for Product and Impurity Analysis

### For Analysis of, for Example, Hydrocodone and Salts Thereof

Product impurity profiles may be determined using a Water Acquity UPLC system and the following parameters: (a) Column: Waters XBridge Shield RP18, 150×3.0 mm, 3.5 μm (PART #186003041) column or equivalent; (b) Injection Volume: 5 μL; (c) Column Temperature: 40° C.; (d) Detector Wavelength: 284 mm; (e) Flow Rate: 0.65 mL/min; (I) Run Time: 30 Min; and using the Gradient Timetable in Table 8, below.

TABLE 8
Gradient Timetable

Mobile
Phase

| Time (min.) | % A | % B | Gradient Change |
|---|---|---|---|
| 0.0 | 90 | 10 | N/A |
| 10.0 | 70 | 30 | Linear |
| 25.0 | 45 | 55 | Linear |
| 25.1 | 90 | 10 | Linear |
| 30.0 | 90 | 10 | Linear |

Mobile Phase A was 1.32 g/L ammonium formate, pH 9.6 adjusted with ammonium hydroxie, prepared by dissolving 1.32±0.01 g of ammonium formate in 1 L of water and mixing thoroughly. Mobile Phase A was then filtered through a pre-conditioned Mobile Phase Filtration Cartridge (3M Empore™ SDB-XC, PN2240 or 3M Empore™ SDB-RPS Extraction Disk Cartridge PN2241). After filtering, the pH of Mobile Phase A was adjusted to pH 9.6 by addition of 28% ammonium hydroxide, as needed. (NOTE: Mobile Phase A may alternatively be the mixed phosphate buffer in deionized water as described in Examples 7-9 above)

Mobile Phase B was HPLC Grade (or purer) acetonitrile.

Retention times for selected products and impurities using the method described above, and Gradient Table 8 were as listed in Table 9, below.

TABLE 9
Retention Times using Gradient Table 8

| | | Retention Time (min) |
|---|---|---|
| | Retention Time | Relative to Hydrocodone |
| Analyte | (min) | Bitartrate |
| Hydrocodone Bitartrate | 11.90 | 1.0 |
| 6β-Tetrahydrothebaine | 14.99 | 1.26 |
| Bitartrate | | |
| Dihydrothebaine Bitartrate | 11.41 | 0.96 |

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A process for the preparation of oxymorphone freebase comprising the step of: 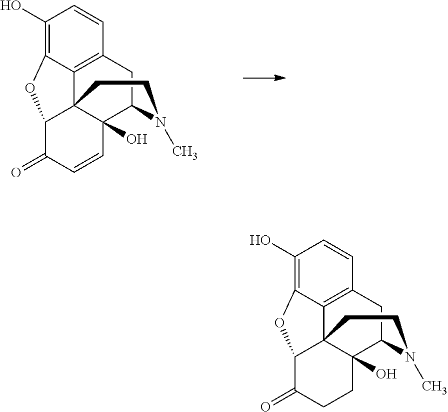
reacting 14-hydroxymorphinone with a hydrogenating agent; optionally in the presence of ethylenediaminetetracetic acid disodium salt; optionally in the presence of a proton source; in dimethylformamide; to yield oxymorphone as a freebase.

2. A process as in claim 1, wherein the hydrogenating agent is hydrogen gas and wherein the 14-hydroxymorphinone is reacted with the hydrogen gas in the presence of Pd/C catalyst.

3. A process as in claim 1, wherein the hydrogenating agent is hydrogen gas; wherein the hydrogen gas is present at a pressure in the range of from about 20 to about 50 psi; wherein the 14-hydroxymorphinone is reacted with the hydrogen gas in the presence of 5% Pd/C catalyst; and wherein the 5% Pd/C catalyst is present in an amount in the range of from about 1 wt % to about 2 wt %.

4. A process as in claim 1, wherein the hydrogenating agent is hydrogen gas; wherein the hydrogen gas is present at a pressure of about 35 psi; wherein the 14-hydroxymorphinone is reacted with the hydrogen gas in the presence of 5% Pd/C catalyst; and wherein the 5% Pd/C catalyst is present in an amount of about 1.8 wt %.

5. A process as in claim 1, wherein the 14-hydroxymorphinone is reacted in the presence of a proton source; and wherein the proton source is dibasic potassium phosphate.

6. A process as in claim 1, wherein the 14-hydroxymorphinone is reacted in the presence of a proton source; wherein the proton source is dibasic potassium phosphate; and wherein the dibasic potassium phosphate is present in an amount in the range of from about 0.005 to about 0.05 kg/kg relative to the amount of 14-hydroxymorphinone.

7. A process as in claim 1, wherein the 14-hydroxymorphinone is reacted in the presence of a proton source; wherein the proton source is dibasic potassium phosphate; and wherein the dibasic potassium phosphate is present in an amount of about 0.01 kg/kg relative to the amount of 14-hydroxymorphinone.

8. A process as in claim 1, wherein the dimethylformamide is present in an amount in the range of from about 1 L/kg to about 5 L/kg relative to the amount of 14-hydroxymorphinone.

9. A process as in claim 1, wherein the dimethylformamide is present in an amount of about 3 L/kg relative to the amount of 14-hydroxymorphinone.

10. A process as in claim 1, wherein the 14-hydroxymorphinone is reacted with the hydrogenating agent at a temperature is in a range of about 20° C. to about 50° C.

11. A process as in claim 1, wherein the 14-hydroxymorphinone is reacted with the hydrogenating agent at a temperature of about 35° C.

12. A process as in claim 1, further comprising precipitating the oxymorphone freebase by addition of water; and isolating the oxymorphone freebase precipitate as a solid.

13. A process as in claim 12, wherein the water is added in an amount in the range of from about 6 L/kg to about 12 L/kg relative to the amount of 14-hydroxymorphinone.

14. A process as in claim 12, wherein the water is added in an amount of about 10 L/kg relative to the amount of 14-hydroxymorphinone.

15. A product prepared according to the process of claim 1.

16. A process for the preparation of oxymorphone freebase comprising the steps of
(a) charging 14-hydroxymorphinone, dibasic potassium phosphate, DMF and 5% Pd/C and optionally EDTA, to a nitrogen purged hydrogenation vessel;
wherein the dibasic potassium phosphate is added in an amount of about 0.01 kg/kg relative to the amount of 14-hydroxymorphinone; wherein the DMF is added in an amount of about 3 L/kg relative to the amount of 14-hydroxymorphinone; and wherein the 5% Pd/C is added in an amount of about 0.018 kg/kg relative to the amount of 14-hydroxymorphinone;
(b) hydrogenating the 14-hydroxymorphinone with H₂ gas at 35 psi and 35° C.; to yield a reaction mixture comprising oxymorphone freebase;
wherein the hydrogenation is continued until greater than about 95% of the 14-hydroxymorphinone is consumed;
(c) filtering the reaction mixture to remove the Pd/C catalyst; and yield a filtrate comprising oxymorphone freebase;
(d) adding water to the filtrate; to yield oxymorphone freebase as a precipitate; wherein the water is added in an amount of about 10 L/kg relative to the amount 14-hydroxymorphinone; and
(e) isolating the oxymorphone freebase as a solid.

17. A product prepared according to the process of claim 16.

18. A process as in claim 1, further comprising the step of: 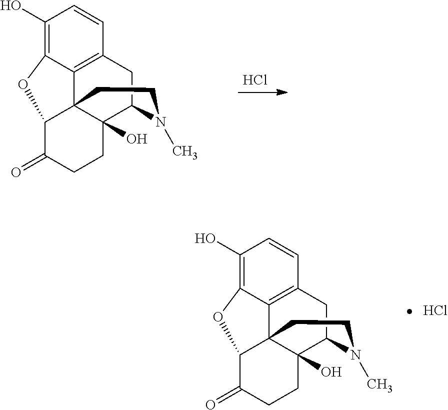
reacting the oxymorphone base with hydrochloric acid, to yield the corresponding oxymorphone hydrochloric acid addition salt.

19. A product prepared according to the process of claim 18.

20. A process as in claim 1 further comprising the step of: 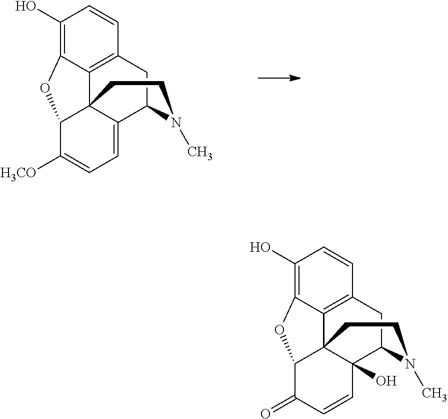
reacting CPS oripavine to yield 14-hydroxymorphinone.

21. A process as in claim 20, wherein the CPS oripavine is reacted with a peroxyacid; in water; to yield 14-hydroxymorphinone.

22. A process as in claim 21, wherein the peroxyacid is prepared in situ by reacting a peroxyacid forming agent with hydrogen peroxide.

23. A process as in claim 22, wherein the peroxyacid forming agent is 90% formic acid; and wherein the 90% formic acid is present in an amount in the range of about 0.1 kg/kg to about 2 kg/kg relative to the amount of CPS oripavine; and wherein the hydrogen peroxide is present in an amount 0.5 molar equivalents to about 3 molar equivalents.

24. A process as in claim 22, wherein the peroxyacid forming agent is 90% formic acid; and wherein the 90% formic acid is present in an amount of in the range of about 1 kg/kg to about 1.1 kg/kg relative to the amount of CPS oripavine; and wherein the hydrogen peroxide is present in an amount 1.05 molar equivalents to about 1.2 molar equivalents.

25. A process as in claim 21, wherein the water is present in an amount in the range of from about 0.1 kg/kg to about 5 kg/kg relative to the amount of CPS oripavine.

26. A process as in claim 21, wherein the water is present in an amount in the range of from about 1.4 kg/kg to about 1.6 kg/kg, relative to the amount of CPS oripavine.

27. A process as in claim 21, wherein the CPS oripavine is reacted with the peroxyacid at a temperature in the range of from about 20° C. to about 28° C.

28. A process as in claim 20, wherein the 14-hydroxymorphinone is precipitated by addition of an organic amine base; and isolated by filtration.

29. A process as in claim 28, wherein the organic base amine is triethylamine; and wherein the triethylamine is added in an amount sufficient to adjust the pH of the reaction mixture to a pH in the range of from about pH 8 to abut pH 10.

30. A process as in claim 20, wherein the 14-hydroxymorphinone is isolated as a solid; wherein the isolated solid comprises less than about 5 wt % impurities.

31. A product prepared according to the process of claim 20.

32. A process as in claim 1, further comprising 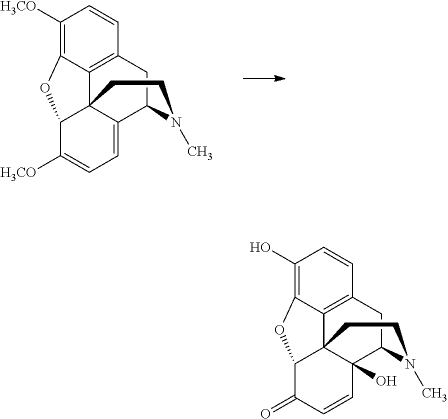
reacting thebaine to yield 14-hydroxymorphinone; and

33. A product prepared according to the process of claim 32.

34. A process as in claim 20 or claim 32, further comprising 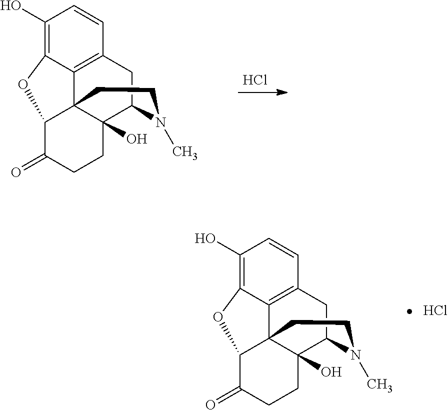
reacting the oxymorphone base with hydrochloric acid, to yield the corresponding oxymorphone hydrochloric acid addition salt.

35. A product prepared according to the process of claim 34.

36. Oxymorphone freebase or oxymorphone hydrochloride; wherein the individual wt % of one or more of (a) 14-hydroxymorphinone, (b) 14-hydroxy-dihydromorphine (α), (c) 8-hydroxyoxymorphone, or (d) 2,2′-bis-oxymorphone is individually less than or equal to about 0.5 wt %.

37. Oxymorphone freebase or oxymorphone hydrochloride; wherein the individual wt % of one or more of (a) 14-hydroxymorphinone, (b) 14-hydroxy-dihydromorphine (α), (c) 8-hydroxyoxymorphone, or (d) 2,2-bis-oxymorphone is individually less than or equal to about 0.1 wt %.

38. An HPLC or UPLC system for analyzing the purity or impurity profile of an opioid compound comprising
(a) a HPLC or UPLC column for receiving mobile phases;
(b) a Mobile Phase A and a Mobile Phase B, wherein the Mobile Phase A and/or Mobile Phase B comprises a buffer and further wherein at least one of the mobile phases has (or is buffered to have) a pH equal to or within 0.5 units of the pK of the opioid compound to be analyzed.

39. A system as in claim 38, wherein the opioid compound is selected from the group consisting of oxymorphone, oxycodone, hydrocodone, hydromorphone, buprenorphine, morphine, codeine, benzhydrocodone, and pharmaceutically acceptable salts thereof.

40. A system as in claim 38, wherein the opioid compound is selected from the group consisting of oxmorphone freebase, oxymorphone hydrochloride, oxycodone, oxycodone hydrochloride, hydromorphone, hydrocodone bitartrate and benzhydrocodone.

41. A system as in claim 38, wherein the Mobile Phase A is a mixed phosphate buffer in water, wherein the pH of the mixed phosphate buffer is in the range of from about pH 9.3 to about pH 9.4; or an ammonium formate buffer in water, wherein the pH of the ammonium formate buffer is about pH 9.6.

42. An HPLC or UPLC method for analyzing the purity or impurity profile of an opioid compound, comprising the step of applying at least one mobile phase to an HPLC or UPLC column, wherein at least one of the mobile phases has (or is buffered to have) a pH equal to or within 0.5 units of the pK of the opioid compound to be analyzed.

43. An HPLC and UPLC method for analyzing an opioid compound comprising the steps of:
(a) selecting a Mobile Phase A and a Mobile Phase B; wherein Mobile Phase A is a mixed phosphate buffer in water or an ammonium formate buffer in water and wherein Mobile Phase B is acetonitrile; and
(b) applying Mobile Phase A and Mobile Phase B with a gradient timetable selected to achieve separation of the opioid compound from any impurities present in the sample.

44. An HPLC or UPLC method for analyzing the purity or impurity profile of an opioid compound comprising the steps of:
(a) injecting a sample of an opioid compound into an HPLC or UPLC column;
(b) applying a Mobile Phase A and a Mobile Phase B to the column; wherein Mobile Phase A is a mixed phosphate buffer in water or an ammonium formate buffer in water; wherein the pH of the mixed phosphate buffer in water is in the range of from about pH 8 to about pH 9; and wherein the pH of the ammonium formate buffer in water is in the range of from about pH 9 to about pH 10; and wherein Mobile Phase B is acetonitrile; and further wherein the Mobile Phase A and the Mobile Phase B are applied with a Mobile Phase gradient selected to separate the opioid compound peaks from the impurity peaks.

45. A method as in claim 43, wherein the Mobile Phase gradient is selected from the group consisting of
Gradient Timetable A:
Gradient Timetable A
Mobile
Phase

| Time (min.) | % A | % B | Curve |
|---|---|---|---|
| Initial | 95 | 5 | Initial |
| 1.00 | 84 | 16 | 6 |
| 2.00 | 84 | 16 | 6 |
| 4.00 | 70 | 30 | 9 |
| 6.00 | 65 | 35 | 9 |
| 8.50 | 45 | 55 | 3 |
| 10.00 | 95 | 5 | 1 |

Gradient Table B:
Gradient Timetable B
Mobile
Phase

| Time (min.) | % A | % B | Gradient Change |
|---|---|---|---|
| 0.00 | 96 | 4 | N/A |
| 1.00 | 86 | 14 | Linear |
| 3.00 | 76 | 24 | Linear |
| 8.00 | 70 | 30 | Linear |
| 12.00 | 70 | 30 | Linear |
| 18.00 | 60 | 40 | Linear |
| 21.00 | 40 | 60 | Linear |
| 26.00 | 40 | 60 | Linear |
| 26.10 | 96 | 4 | Step @26 minutes |
| 30.00 | 96 | 4 | Linear |

Gradient Table C:
Gradient Timetable C
Mobile
Phase

| Time (min.) | % A | % B | Gradient Change |
|---|---|---|---|
| Initial | 96 | 4 | N/A |
| 0.30 | 86 | 14 | Linear |
| 0.85 | 76 | 24 | Linear |
| 2.40 | 70 | 30 | Linear |
| 3.70 | 70 | 30 | Linear |
| 5.00 | 60 | 40 | Linear |
| 6.50 | 40 | 60 | Linear |
| 7.50 | 40 | 60 | Linear |
| 9.00 | 96 | 4 | Ste @ 7.5 min |

and Gradient Table D:
Gradient Table D
Mobile
Phase

| Time (min.) | % A | % B | Gradient Change |
|---|---|---|---|
| 0.0 | 90 | 10 | N/A |
| 10.0 | 70 | 30 | Linear |
| 25.0 | 45 | 55 | Linear |
| 25.1 | 90 | 10 | Linear |
| 30.0 | 90 | 10 | Linear |
